(19) United States
(12) Patent Application Publication
(10) Pub. No.: US 2021/0299438 A1
(43) Pub. Date: Sep. 30, 2021

(54) Alopecia Repair System

(71) Applicant: Neurostim Technologies LLC, Waltham, MA (US)

(72) Inventors: Hoo-min D. TOONG, Cambridge, MA (US); William C. ALTMANN, Austin, TX (US); Constance CARDASIS, Jupiter, FL (US); Ann G. YEE, Brookline, MA (US); Pheng DANG, San Jose, CA (US)

(21) Appl. No.: 17/210,685

(22) Filed: Mar. 24, 2021

Related U.S. Application Data
(60) Provisional application No. 62/994,199, filed on Mar. 24, 2020.

(51) Int. Cl. A61N 1/32; A61N 1/04; A61N 1/36

(52) U.S. Cl. CPC A61N 1/325; A61N 1/36031; A61N 1/0428

(57) ABSTRACT

Example inventions are directed to reducing hair loss in a target follicle area where the hair loss has occurred. Examples affix a patch externally on a dermis of a user so that the patch extends over the target follicle area of the user, the patch comprising a flexible substrate, a processor directly coupled to the substrate, and electrodes directly coupled to the substrate. Examples activate the patch to initiate a treatment session, the activating comprising generating electrical stimuli via the electrodes that is directed at the target follicle area.

### CROSS REFERENCE TO RELATED APPLICATIONS

This application claims priority to U.S. Provisional Patent Application Ser. No. 62/994,199 filed on Mar. 24, 2020, the disclosure of which is hereby incorporated by reference.

### FIELD

Example inventions are directed to systems and methods for reducing hair loss due to alopecia by treating the skin with transcutaneous electrical stimulation.

### BACKGROUND INFORMATION

Hair loss from alopecia is caused by dysfunction in the tissues associated with the hair follicles, and other changes in the skin, particularly as one ages. Alopecia has several different forms: Androgenetic, or male pattern baldness, but also in women; Areata, a type prevalent in women; Telogen Effluvium; Traction Alopecia; Alopecia Universalis; and several others. The epidemiology has been well documented as well as the etiology. Two current FDA approved medications that are effective in halting the progression of hair loss are minoxidil (Rogaine 2%/5%), proven to increase local blood flow in the scalp, and finasteride, which suppresses the level of Dihydrotestosterone (“DHT”), an androgen, which is one of the associated causes of alopecia in those individuals genetically predisposed to alopecia.

### BRIEF DESCRIPTION OF THE DRAWINGS

FIG. 1 illustrates an example patch that is affixed to a location behind an ankle bone of a user at a follicle loss area.

FIG. 2 is a block diagram illustrating hardware/software related elements of an example of the patch of FIG. 1.

FIG. 3A is a circuit diagram of an example of a boosted voltage circuit that provides feedback.

FIG. 3B is a circuit diagram of an example of a charge application circuit that uses an output of the boosted voltage circuit.

FIG. 4 is a flow diagram of the functionality of the controller of monitoring and controlling the output voltage, including its ramp rate.

FIG. 5 is a flow diagram in accordance with one example of an adaptive protocol.

FIG. 6 is a Differential Integrator Circuit used in the adaptive protocol in accordance with one example.

FIG. 7 is a table relating charge duration vs. frequency to provide feedback to the adaptive protocol in accordance with one example.

FIG. 8 is an illustration of components of an alopecia repair system in accordance with example inventions.

FIG. 9 illustrates a user with skin having epidermis, dermis and a follicle area to be repaired.

FIG. 10 illustrates another example of the alopecia repair system.

FIG. 11 illustrates example stimulation waveforms for repairing alopecia in accordance with example inventions.

FIG. 12 illustrates the patch with multiple electrodes that are adapted to provide both stimulation and sensing in accordance with example inventions.

FIG. 13 illustrates a stack-up view of the patch accordance to example inventions.

FIG. 14 illustrates a sectional view of the patch affixed to a dermis in accordance to example inventions.

### DETAILED DESCRIPTION

A non-invasive nerve/tissue patch/activator in accordance with various examples disclosed herein includes novel circuitry to adequately boost voltage to a required level and to maintain a substantially constant level of charge for nerve activation or tissue stimulation. Optionally, a feedback loop provides for an automatic determination and adaptation of the applied charge level. In example inventions, the patch is used facilitate the repair of hair loss due to alopecia, with or without the use of medications. An integrated system is placed on the skin of a user and can be activated, adjusted and used with or without the help of a medical professional. The integrated system includes hardware and software to monitor biometrics related to alopecia repair, and to stimulate the skin to continue the repair process, while also providing a closed-loop system.

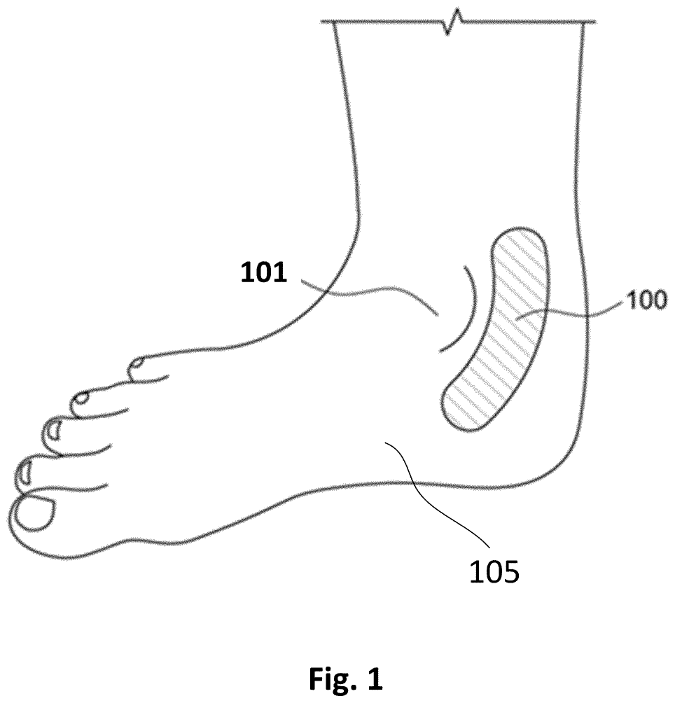

FIG. 1 illustrates an example patch 100, also referred to as a smart band aid or smartpad or Topical Nerve/Tissue Activator (“TNA”) or topical nerve activation and tissue stimulation patch, that is affixed to a location behind an ankle bone 101 of a user 105 in one example use, but can be affixed in any other location where alopecia needs to be repaired, generally on the user's head.

Patch 100 is used to stimulate nerves and tissues and is convenient, unobtrusive, self-powered, and may be controlled from a smartphone or other control device. This has the advantage of being non-invasive, controlled by consumers themselves, and potentially distributed over the counter without a prescription. Patch 100 provides a means of stimulating nerves and tissues without penetrating the dermis, and can be applied to the surface of the dermis at a location appropriate for the hair loss of interest. In examples, patch 100 is applied by the user and is disposable.

Patch 100 in examples can be any type of device that can be fixedly attached to a user, using adhesive in some examples, and includes a processor/controller and instructions that are executed by the processor, or a hardware implementation without software instructions, as well as electrodes that apply an electrical stimulation to the surface of the user's skin, and associated electrical circuitry. Patch 100 in one example provides topical nerve or tissue activation/stimulation on the user to provide benefits to the user, including bladder management for an overactive bladder (“OAB”) or alopecia repair.

Patch 100 in one example can include a flexible substrate, a malleable dermis conforming bottom surface of the substrate including adhesive and adapted to contact the dermis, a flexible top outer surface of the substrate approximately parallel to the bottom surface, a plurality of electrodes positioned on the patch proximal to the bottom surface and located beneath the top outer surface and directly contacting the flexible substrate, electronic circuitry (as disclosed herein) embedded in the patch and located beneath the top outer surface and integrated as a system on a chip that is directly contacting the flexible substrate, the electronic circuitry integrated as a system on a chip or discrete components and including an electrical signal generator integral to the malleable dermis conforming bottom surface configured to electrically activate the one or more electrodes, a signal activator coupled to the electrical signal generator, a nerve/tissue stimulation sensor that provides feedback in response to a stimulation of one or more nerves or tissues, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to stimulate one or more nerves or tissues of a user wearing patch 100 at least at one location proximate to patch 100. Additional details of examples of patch 100 beyond the novel details disclosed herein are disclosed in U.S. Pat. No. 10,016,600, entitled “Topical Neurological Stimulation”, the disclosure of which is hereby incorporated by reference.

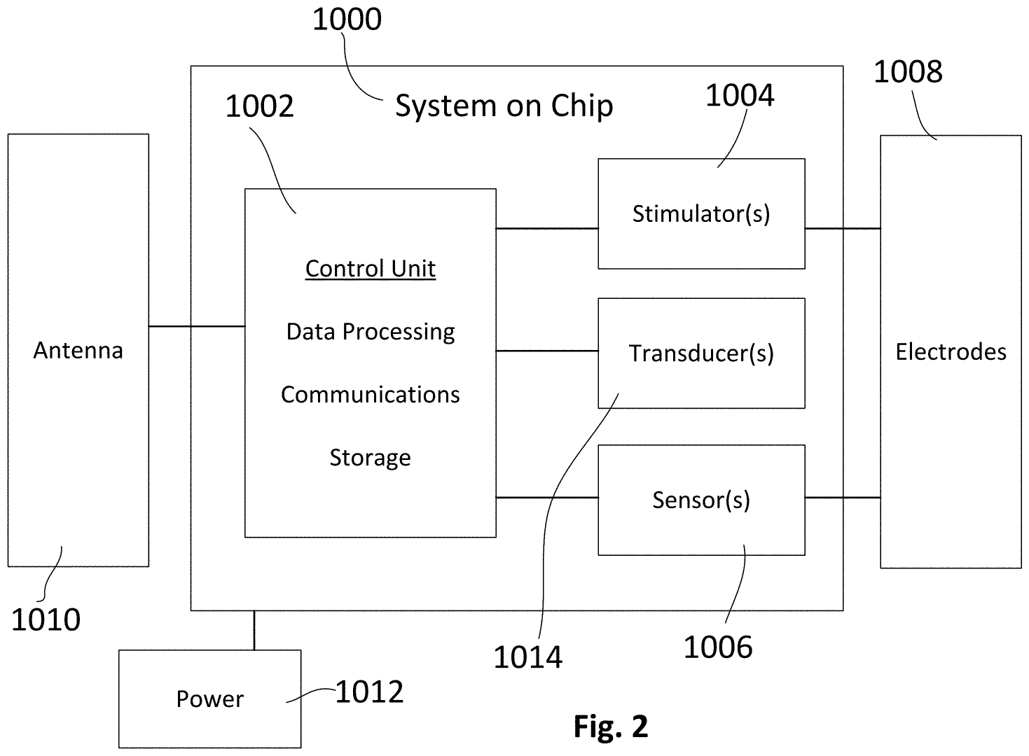

FIG. 2 is a block diagram illustrating hardware/software related elements of an example of patch 100 of FIG. 1. Patch 100 includes electronic circuits or chips 1000 that perform the functions of: communications with an external control device, such as a smartphone or fob, or external processing such as cloud based processing devices, nerve and tissue activation via electrodes 1008 that produce a wide range of electric fields according to a treatment regimen, and a wide range of sensors 1006 such as, but not limited to, mechanical motion and pressure, temperature, humidity, acoustic, chemical and positioning sensors. In another example, patch 100 includes transducers 1014 to transmit signals to the tissue or to receive signals from the tissue.

One arrangement is to integrate a wide variety of these functions into a system on a chip 1000. Within this is shown a control unit 1002 for data processing, communications, transducer interface and storage, and one or more stimulators 1004 and sensors 1006 that are connected to electrodes 1008. Control unit 1002 can be implemented by a general purpose processor/controller, or a specific purpose processor/controller, or a special purpose logical circuit. An antenna 1010 is incorporated for external communications by control unit 1002. Also included is an internal power supply 1012, which may be, for example, a battery. Other examples may include an external power supply. It may be necessary to include more than one chip to accommodate a wide range of voltages for data processing and stimulation. Electronic circuits and chips will communicate with each other via conductive tracks within the device capable of transferring data and/or power.

Patch 100 interprets a data stream from control unit 1002 to separate out message headers and delimiters from control instructions. In one example, control instructions include information such as voltage level and pulse pattern. Patch 100 activates stimulator 1004 to generate a stimulation signal to electrodes 1008 placed on the tissue according to the control instructions. In another example, patch 100 activates transducer 1014 to send a signal to the tissue. In another example, control instructions cause information such as voltage level and a pulse pattern to be retrieved from a library stored by patch 100, such as storage within control unit 1002.

Patch 100 receives sensory signals from the tissue and translates them to a data stream that is recognized by control unit 1002. Sensory signals can include electrical, mechanical, acoustic, optical and chemical signals. Sensory signals are received by patch 100 through electrodes 1008 or from other inputs originating from mechanical, acoustic, optical, or chemical transducers. For example, an electrical signal from the tissue is introduced to patch 100 through electrodes 1008, is converted from an analog signal to a digital signal and then inserted into a data stream that is sent through antenna 1010 to the external control device. In another example, an acoustic signal is received by transducer 1014, converted from an analog signal to a digital signal and then inserted into a data stream that is sent through the antenna 1010 to the external control device. In another example a pH sensor will detect the user's skin pH or the pH of a formulation included with the patch and sends that data to the control unit. In some examples, sensory signals from the tissue are directly interfaced to the external control device for processing.

In examples of patch 100 disclosed above, when being used for therapeutic treatment such as bladder management for OAB or alopecia repair, there is a need to control the voltage by boosting the voltage to a selected level and providing the same level of charge upon activation to a mammalian nerve or tissue. Further, there is a need to conserve battery life by selectively using battery power. Further, there is a need to create a compact electronics package to facilitate mounting the electronics package on a relatively small mammalian dermal patch in the range of the size of an ordinary band aid.

Adaptive Circuit

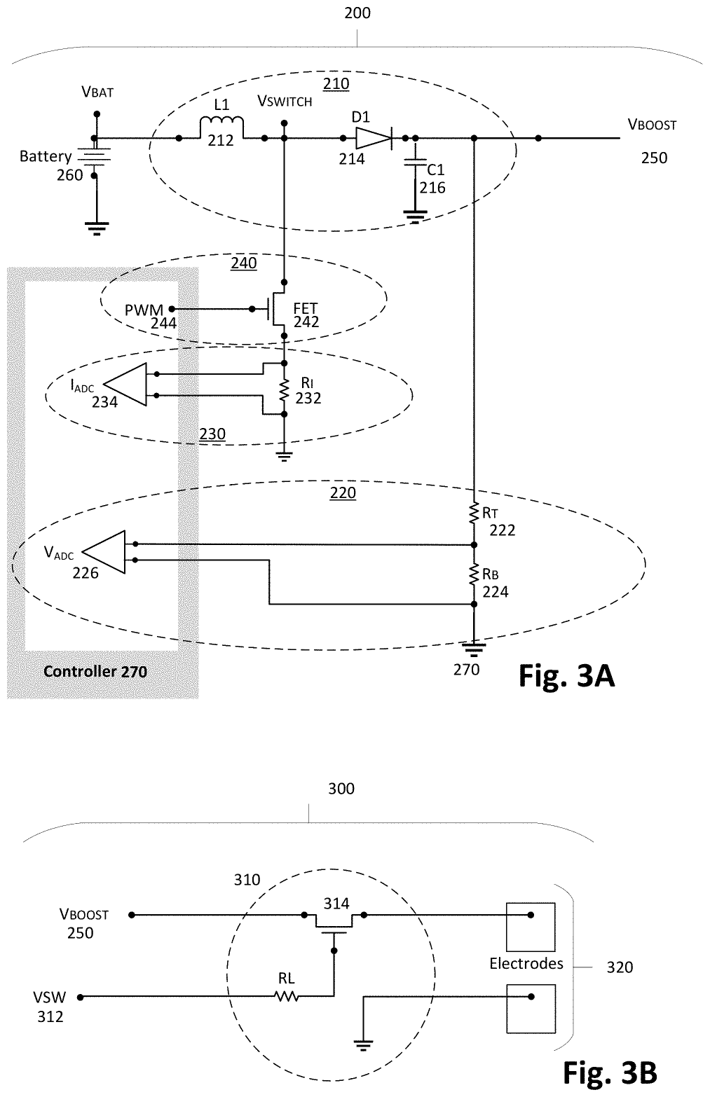

To meet the above needs, examples implement a novel boosted voltage circuit that includes a feedback circuit and a charge application circuit. FIG. 3A is a circuit diagram of an example of the boosted voltage circuit 200 that provides feedback. FIG. 3B is a circuit diagram of an example of a charge application circuit 300 that uses an output of boosted voltage circuit 200. Boosted voltage circuit 200 includes both electrical components and a controller/processor 270 that includes a sequence of instructions that together modify the voltage level of activation/stimulation delivered to the external dermis of user 105 by patch 100 through electrodes. Controller/processor 270 in examples implements control unit 1002 of FIG. 2.

Boosted voltage circuit 200 can replace an independent analog-controlled boost regulator by using a digital control loop to create a regulated voltage, output voltage 250, from the battery source. Output voltage 250 is provided as an input voltage to charge application circuit 300. In examples, this voltage provides nerve stimulation currents through the dermis/skin to deliver therapy for repairing hair loss. Output voltage 250, or “V_{Boost}”, at voltage output node 250, uses two digital feedback paths 220, 230, through controller 270. In each of these paths, controller 270 uses sequences of instructions to interpret the measured voltages at voltage monitor 226, or “V_{ADC}” and current monitor 234, or “I_{ADC}”, and determines the proper output control for accurate and stable output voltage 250.

Boosted voltage circuit 200 includes an inductor 212, a diode 214, a capacitor 216 that together implement a boosted converter circuit 210. A voltage monitoring circuit 220 includes a resistor divider formed by a top resistor 222, or “RT”, a bottom resistor 224, or “R_{B}” and voltage monitor 226. A current monitoring circuit 230 includes a current measuring resistor 232, or “R_{I}” and current monitor 234. A pulse width modulation (“PWM”) circuit 240 includes a field-effect transistor (“FET”) switch 242, and a PWM driver 244. Output voltage 250 functions as a sink for the electrical energy. An input voltage 260, or “V_{BAT}”, is the source for the electrical energy, and can be implemented by power 1012 of FIG. 2.

PWM circuit 240 alters the “on” time within a digital square wave, fixed or variable frequency signal to change the ratio of time that a power switch is commanded to be “on” versus “off.” In boosted voltage circuit 200, PWM driver 244 drives FET switch 242 to “on” and “off” states.

In operation, when FET switch 242 is on, i.e., conducting, the drain of FET switch 242 is brought down to Ground/GND or ground node 270. FET switch 242 remains on until its current reaches a level selected by controller 270 acting as a servo controller. This current is measured as a representative voltage on current measuring resistor 232 detected by current monitor 234. Due to the inductance of inductor 212, energy is stored in the magnetic field within inductor 212. The current flows through current measuring resistor 232 to ground until FET switch 242 is opened by PWM driver 244.

When the intended pulse width duration is achieved, controller 270 turns off FET switch 242. The current in inductor 212 reroutes from FET switch 242 to diode 214, causing diode 214 to forward current. Diode 214 charges capacitor 216. Therefore, controller 270 controls the voltage level at capacitor 216.

Output voltage 250 is controlled using an outer servo loop of voltage monitor 226 and controller 270. Output voltage 250 is measured by the resistor divider using top resistor 222, bottom resistor 224, and voltage monitor 226. The values of top resistor 222 and bottom resistor 224 are selected to keep the voltage across bottom resistor 224 within the monitoring range of voltage monitor 226. Controller 270 monitors the output value from voltage monitor 226.

Charge application circuit 300 includes a pulse application circuit 310 that includes an enable switch 314. Controller 270 does not allow enable switch 314 to turn on unless output voltage 250 is within a desired upper and lower range of the desired value of output voltage 250. Pulse application circuit 310 is operated by controller 270 by asserting an enable signal 312, or “VSW”, which turns on enable switch 314 to pass the electrical energy represented by output voltage 250 through electrodes 320. At the same time, controller 270 continues to monitor output voltage 250 and controls PWM driver 244 to switch FET switch 242 on and off and to maintain capacitor 216 to the desired value of output voltage 250.

The stability of output voltage 250 can be increased by an optional inner feedback loop through FET Switch 242, current measuring resistor 232, and current monitor 234. Controller 270 monitors the output value from current monitor 234 at a faster rate than the monitoring on voltage monitor 226 so that the variations in the voltages achieved at the cathode of diode 214 are minimized, thereby improving control of the voltage swing and load sensitivity of output voltage 250.

In one example, a voltage doubler circuit is added to boosted voltage circuit 200 to double the high voltage output or to reduce voltage stress on FET 242. The voltage doubler circuit builds charge in a transfer capacitor when FET 242 is turned on and adds voltage to the output of boosted voltage circuit 200 when FET 242 is turned off.

As described, in examples, controller 270 uses multiple feedback loops to adjust the duty cycle of PWM driver 244 to create a stable output voltage 250 across a range of values. Controller 270 uses multiple feedback loops and monitoring circuit parameters to control output voltage 250 and to evaluate a proper function of the hardware. Controller 270 acts on the feedback and monitoring values in order to provide improved patient safety and reduced electrical hazard by disabling incorrect electrical functions.

In some examples, controller 270 implements the monitoring instructions in firmware or software code. In some examples, controller 270 implements the monitoring instructions in a hardware state machine.

In some examples, voltage monitor 226 is an internal feature of controller 270. In some examples, voltage monitor 226 is an external component, which delivers its digital output value to a digital input port of controller 270.

In some examples, current monitor 234 is an internal feature of controller 270. In some examples, current monitor 234 is an external component, which delivers its digital output value to a digital input port of controller 270.

An advantage of boosted voltage circuit 200 over known circuits is decreased component count which may result in reduced costs, reduced circuit board size and higher reliability. Further, boosted voltage circuit 200 provides for centralized processing of all feedback data which leads to faster response to malfunctions. Further, boosted voltage circuit 200 controls outflow current from V_{BAT} 260, which increases the battery's lifetime and reliability.

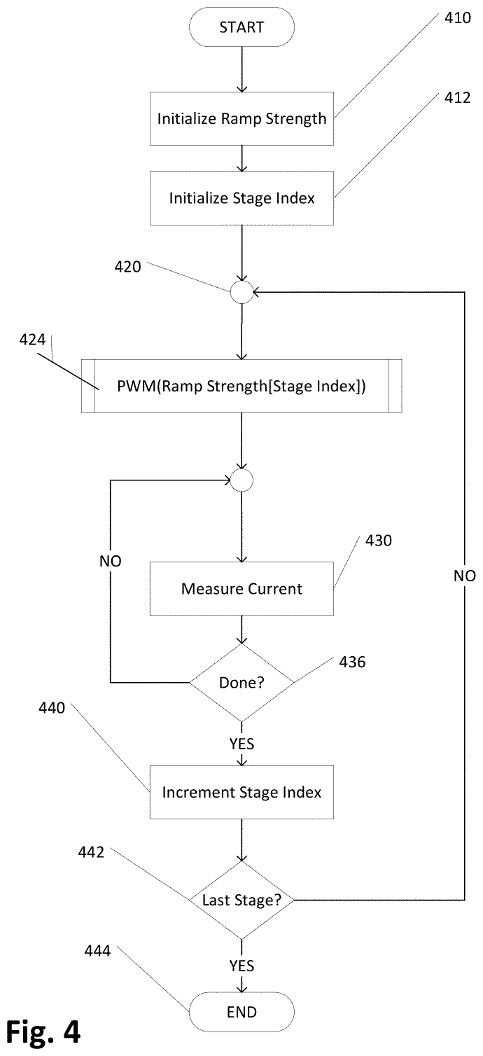

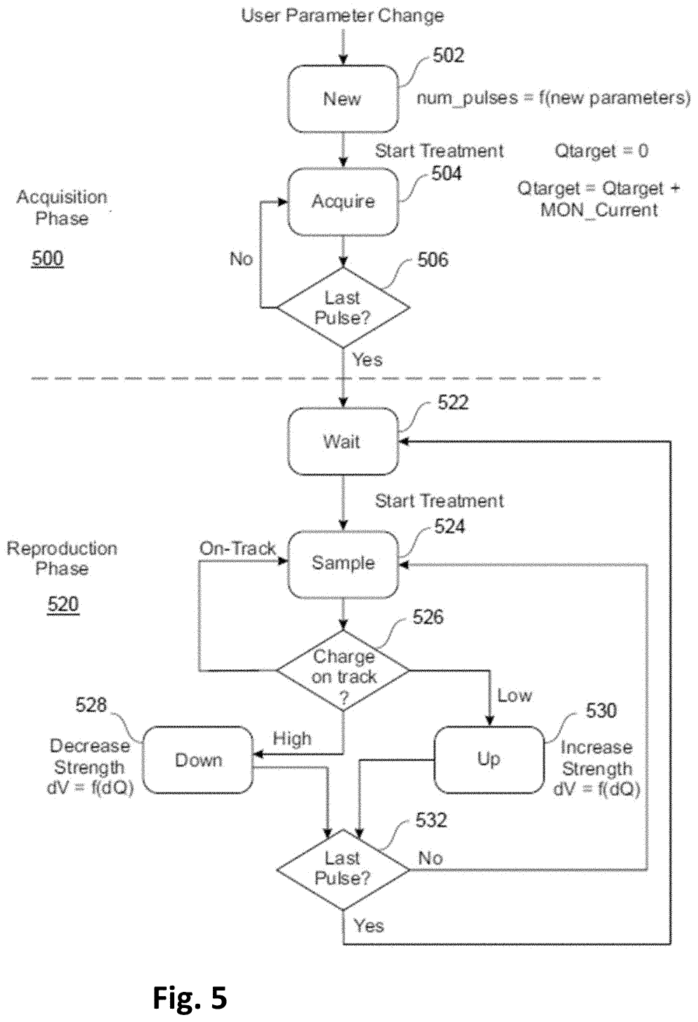

FIG. 4 is a flow diagram of the functionality of controller 270 of monitoring and controlling output voltage 250, including its ramp rate. In one example, the functionality of the flow diagram of FIG. 4, and FIG. 5 below, is implemented by software stored in memory or other computer readable or tangible medium, and executed by a processor. In other examples, the functionality may be performed by hardware (e.g., through the use of an application-specific integrated circuit (“ASIC”), a programmable gate array (“PGA”), a field programmable gate array (“FPGA”), etc.), or any combination of hardware and software.

The pulse width modulation of FET switch 242 is controlled by one or more pulses for which the setting of each pulse width allows more or less charge to accumulate as a voltage at capacitor 216 through diode 214. This pulse width setting is referred to as the ramp strength and it is initialized at 410. Controller 270 enables each pulse group in sequence with a pre-determined pulse width, one stage at a time, using a stage index that is initialized at 412. The desired ramp strength is converted to a pulse width at 424, which enables and disables FET switch 242 according to the pulse width. During the intervals when FET switch 242 is “on”, the current is measured by current monitor 234 at 430 and checked against the expected value at 436. When the current reaches the expected value, the stage is complete and the stage index is incremented at 440. If the desired number of stages has been applied 442, then the functionality is complete. Otherwise, the functionality continues to the next stage at 420.

As a result of the functionality of FIG. 4, V_{BAT} 260 used in patch 100 operates for longer periods as the current drawn from the battery ramps at a low rate of increase to reduce the peak current needed to achieve the final voltage level 250 for each activation/stimulation treatment. PWM 244 duty cycle is adjusted by controller 270 to change the ramp strength at 410 to improve the useful life of the battery.

An open loop protocol to control current to electrodes in known neural stimulation devices does not have feedback controls. It commands a voltage to be set, but does not check the actual current delivered. A stimulation pulse is sent based on preset parameters and cannot be modified based on feedback from the patient's anatomy. When the device is removed and repositioned, the electrode placement varies. Also the humidity and temperature of the anatomy changes throughout the day. All these factors affect the actual charge delivery if the voltage is preset. Charge control is a patient safety feature and facilitates an improvement in patient comfort, treatment consistency and efficacy of treatment.

In contrast, examples of patch 100 includes features that address these shortcomings using controller 270 to regulate the charge applied by electrodes 320. Controller 270 samples the voltage of the stimulation waveform, providing feedback and impedance calculations for an adaptive protocol to modify the stimulation waveform in real time. The current delivered to the anatomy by the stimulation waveform is integrated using a differential integrator and sampled and then summed to determine the actual charge delivered to the user for a treatment, such as OAB or repairing alopecia. After every pulse in a stimulation event, this data is analyzed and used to modify, in real time, subsequent pulses.

This hardware adaptation allows a firmware protocol to implement the adaptive protocol. This protocol regulates the charge applied to the body by changing output voltage (“V_{BOOST}”) 250. A treatment is performed by a sequence of periodic pulses, which deliver charge into the body through electrodes 320. Some of the parameters of the treatment are fixed and some are user adjustable. The strength, duration and frequency may be user adjustable. The user may adjust these parameters as necessary for comfort and efficacy. The strength may be lowered if there is discomfort and raised if nothing is felt. The duration can be increased if the maximum acceptable strength results in an ineffective treatment.

Adaptive Protocol

A flow diagram in accordance with one example of the adaptive protocol disclosed above is shown in FIG. 5. The adaptive protocol strives to repeatedly and reliably deliver a target charge (“Q_{target}”) in coulombs during a treatment and to account for any environmental changes. Therefore, the functionality of FIG. 5 is to adjust the charge level applied to a user based on feedback, rather than use a constant level. Environmental changes include changes to sensor signals received by the patch.

The mathematical expression of this protocol is as follows: Q_{target}=Q_{target} (A*dS+B*dT), where A is the Strength Coefficient—determined empirically, dS is the user change in Strength, B is the Duration Coefficient—determined empirically, and dT is the user change in Duration.

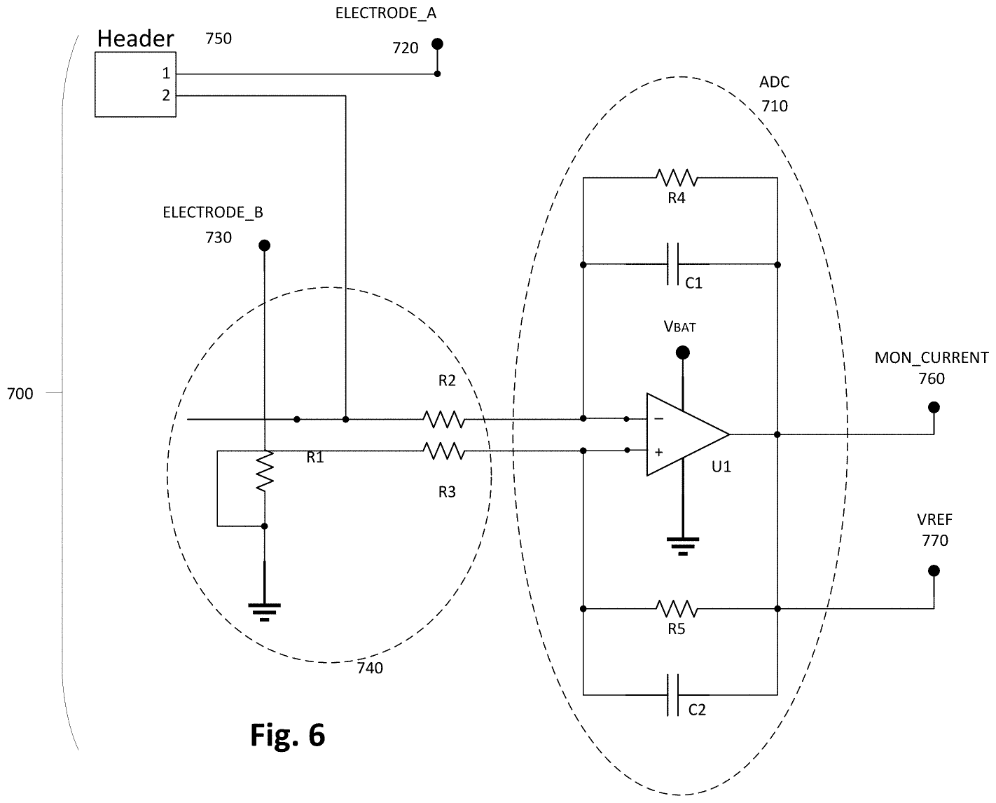

The adaptive protocol includes two phases in one example: Acquisition phase 500 and Reproduction phase 520. Any change in user parameters places the adaptive protocol in the Acquisition phase. When the first treatment is started, a new baseline charge is computed based on the new parameters. At a new acquisition phase at 502, all data from the previous charge application is discarded. In one example, 502 indicates the first time for the current usage where the user places patch 100 on a portion of the body and manually adjusts the charge level, which is a series of charge pulses, until it feels suitable, or any time the charge level is changed, either manually or automatically. The treatment then starts. The mathematical expression of this function of the application of a charge is as follows:

$$The charge delivered in a treatment is {Q}_{target} = {{\sum }^{T * f}}_{i = 1} {Q}_{pulse} ( i )$$

Where T is the duration; f is the count of pulses for one treatment (e.g., Hertz or cycles/second) of “Rep Rate”; Qₚᵤₗₛₑ (i) is the measured charge delivered by Pulse (i) in the treatment pulse train provided as a voltage MON_CURRENT that is the result of a Differential Integrator circuit shown in FIG. 6 (i.e., the average amount of charge per pulse). Differential Integrator circuit 700 of FIG. 6 is an example of a circuit used to integrate current measured over time and quantify the delivered charge and therefore determine the charge output over a treatment pulse. The number of pulses in the treatment is T*f.

As shown in of FIG. 6, MON_CURRENT 760 is the result of the Differential Integrator Circuit 700. The Analog to Digital Conversion (“ADC”) 710 feature is used to quantify voltage into a number representing the delivered charge. The voltage is measured between Electrode A 720 and Electrode B 730, using a Kelvin Connection 740. Electrode A 720 and Electrode B 730 are connected to a header 750. A reference voltage, VREF 770, is included to keep the measurement in range.

In some examples, Analog to Digital Conversion 710 is an internal feature of controller 270. In some examples, Analog to Digital Conversion 710 is an external component, which delivers its digital output value to a digital input port on Controller 270.

At 504 and 506, every pulse is sampled. In one example, the functionality of 504 and 506 lasts for 10 seconds with a pulse rate of 20 Hz. The result of Acquisition phase 500 is the target pulse charge of Q_{target}.

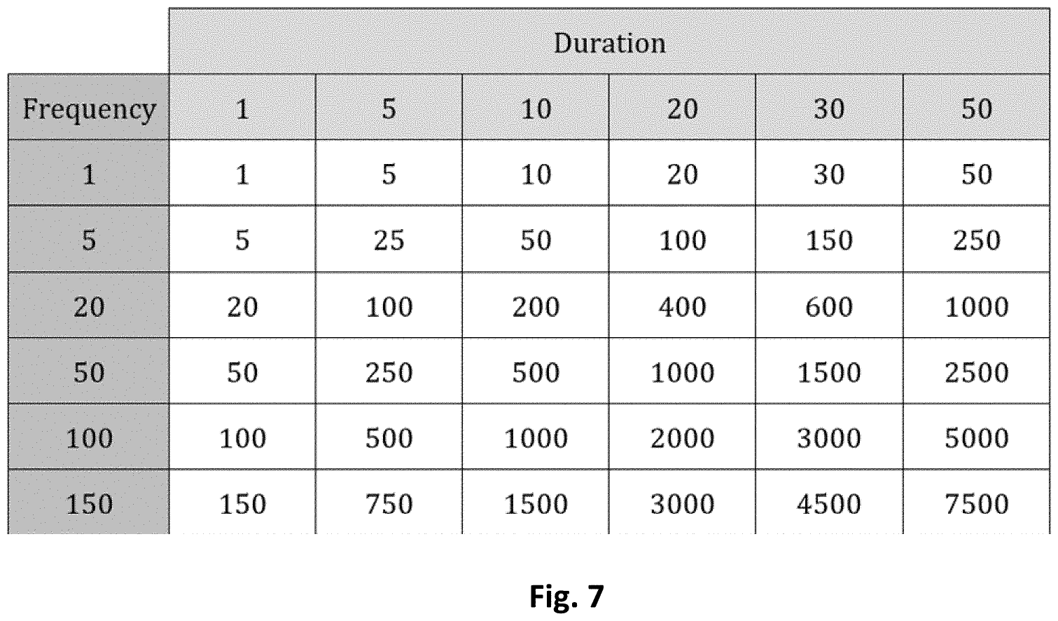

FIG. 7 is a table in accordance with one example showing the number of pulses per treatment measured against two parameters, frequency and duration. Frequency is shown on the Y-axis and duration on the X-axis. The adaptive protocol in general performs better when using more pulses. One example uses a minimum of 100 pulses to provide for solid convergence of charge data feedback, although a less number of pulses can be used in other examples. Referring to the FIG. 7, a frequency setting of 20 Hz and duration of 10 seconds produces 200 pulses. The range of duration of treatment extends to minutes, hours, and in some cases days, with the corresponding large number of pulses applied during that treatment time.

The reproduction phase 520 begins in one example when the user initiates another subsequent treatment after acquisition phase 500 and the resulting acquisition of the baseline charge, Q_{target}. For example, a full treatment cycle, as discussed above, may take 10 seconds. After, for example, a two-hour pause as shown at wait period 522, the user may then initiate another treatment. During this phase, the adaptive protocol attempts to deliver Q_{target} for each subsequent treatment. The functionality of reproduction phase 520 is needed because, during the wait period 522, conditions such as the impedance of the user's body due to sweat or air humidity may have changed. The differential integrator is sampled at the end of each Pulse in the Treatment. At that point, the next treatment is started and the differential integrator is sampled for each pulse at 524 for purposes of comparison to the acquisition phase Q_{target}. Sampling the pulse includes measuring the output of the pulse in terms of total electric charge. The output of the integrator of FIG. 6 in voltage, referred to as Mon_Current 760, is a direct linear relationship to the delivered charge and provides a reading of how much charge is leaving the device and entering the user. At 526, each single pulse is compared to the charge value determined in Acquisition phase 500 (i.e., the target charge) and the next pulse will be adjusted in the direction of the difference.

NUM_PULSES=(T*f)

After each pulse, the observed charge, Qₚᵤₗₛₑ(i), is compared to the expected charge per pulse.

Qₚᵤₗₛₑ(i)>Q_{target}/NUM_PULSES ?

The output charge or “V_{BOOST}” is then modified at either 528 (decreasing) or 530 (increasing) for the subsequent pulse by:

dV(i)=G[Q_{target}/NUM_PULSES−Qₚᵤₗₛₑ(i)]

where G is the Voltage adjustment Coefficient—determined empirically. The process continues until the last pulse at 532.

A safety feature assures that the V_{BOOST} will never be adjusted higher by more than 10%. If more charge is necessary, then the repetition rate or duration can be increased.

In one example a boosted voltage circuit uses dedicated circuits to servo the boosted voltage. These circuits process voltage and/or current measurements to control the PWM duty cycle of the boosted voltage circuit's switch. The system controller can set the voltage by adjusting the gain of the feedback loop in the boosted voltage circuit. This is done with a digital potentiometer or other digital to analog circuit.

In one example, in general, the current is sampled for every pulse during acquisition phase 500 to establish target charge for reproduction. The voltage is then adjusted via a digital potentiometer, herein referred to as “Pot”, during reproduction phase 520 to achieve the established target_charge.

The digital Pot is calibrated with the actual voltage at startup. A table is generated with sampled voltage for each wiper value. Tables are also precomputed storing the Pot wiper increment needed for 1v and 5v output delta at each pot level. This enables quick reference for voltage adjustments during the reproduction phase. The tables may need periodic recalibration due to battery level.

In one example, during acquisition phase 500, the data set=100 pulses and every pulse is sampled and the average is used as the target_charge for reproduction phase 520. In general, fewer pulses provide a weaker data sample to use as a basis for reproduction phase 520.

In one example, during acquisition phase 500, the maximum data set=1000 pulses. The maximum is used to avoid overflow of 32 bit integers in accumulating the sum of samples. Further, 1000 pulses in one example is a sufficiently large data set and collecting more is likely unnecessary.

After 1000 pulses for the above example, the target_charge is computed. Additional pulses beyond 1000 in the acquisition phase do not contribute to the computation of the target charge. In other examples, the maximum data set is greater than 1000 pulses when longer treatment cycle times are desired.

In one example, the first 3-4 pulses are generally higher than the rest so these are not used in acquisition phase 500. This is also accounted for in reproduction phase 520. Using these too high values can result in target charge being set too high and over stimulating on the subsequent treatments in reproduction phase 520. In other examples, more advanced averaging algorithms could be applied to eliminate high and low values.

In an example, there may be a safety concern about automatically increasing the voltage. For example, if there is poor connection between the device and the user's skin, the voltage may auto-adjust at 530 up to the max. The impedance may then be reduced, for example by the user pressing the device firmly, which may result in a sudden high current. Therefore, in one example, if the sample is 500 mv or more higher than the target, it immediately adjusts to the minimum voltage. This example then remains in reproduction phase 520 and should adjust back to the target current/charge level. In another example, the maximum voltage increase is set for a single treatment (e.g., 10V). More than that is not needed to achieve the established target_charge. In another example, a max is set for V_{BOOST} (e.g., 80V).

In various examples, it is desired to have stability during reproduction phase 520. In one example, this is accomplished by adjusting the voltage by steps. However, a relatively large step adjustment can result in oscillation or over stimulation. Therefore, voltage adjustments may be made in smaller steps. The step size may be based on both the delta between the target and sample current as well as on the actual V_{BOOST} voltage level. This facilitates a quick and stable/smooth convergence to the target charge and uses a more gradual adjustments at lower voltages for more sensitive users.

The following are the conditions that may be evaluated to determine the adjustment step.
delta-mon_current=abs(sample_mon_current−target_charge)
If delta_mon_current>500 mv and V_{BOOST}>20V then step=5V for increase adjustments
(For decrease adjustments a 500 mv delta triggers emergency decrease to minimum Voltage)
If delta_mon_current>200 mv then step=1V
If delta_mon_current>100 mv and delta_mon_current>5%*
sample_mon_current then step=1V

In other examples, new treatments are started with voltage lower than target voltage with a voltage buffer of approximately 10%. The impedance is unknown at the treatment start. These examples save the target_voltage in use at the end of a treatment. If the user has not adjusted the strength parameter manually, it starts a new treatment with saved target_voltage with the 10% buffer. This achieves target current quickly with the 10% buffer to avoid possible over stimulation in case impedance has been reduced. This also compensates for the first 3-4 pulses that are generally higher.

As disclosed, examples apply an initial charge level, and then automatically adjust based on feedback of the amount of current being applied. The charge amount can be varied up or down while being applied. Therefore, rather than setting and then applying a fixed voltage level throughout a treatment cycle, implementations of the invention measure the amount of charge that is being input to the user, and adjust accordingly throughout the treatment to maintain a target charge level that is suitable for the current environment.

The Adaptive Circuit described above provides the means to monitor the charge sent through the electrodes to the user's tissue and to adjust the strength and duration of sending charge so as to adapt to changes in the impedance through the electrode-to-skin interface and through the user's tissue such that the field strength at the target nerve or tissue is within the bounds needed to overcome the action potential of that nerve or tissue stimulation threshold at that location and activate a nerve impulse or tissue response. These changes in impedance may be caused by environmental changes, such as wetness or dryness of the skin or underlying tissue, or by applied lotion or the like; or by tissue changes, such as skin dryness; or by changes in the device's placement on the user's skin, such as by removing the patch and re-applying it in a different location or orientation relative to the target nerve or tissue; or by combinations of the above and other factors.

The combined circuits and circuit controls disclose herein generate a charge that is repeated on subsequent uses. The voltage boost conserves battery power by generating voltage on demand. The result is an effective and compact electronics package suitable for mounting on or in a fabric or similar material for adherence to a dermis that allows electrodes to be placed near selected nerves or tissues to be activated or stimulated.

Alopecia Repair

In some example inventions, patch 100, disclosed above, is used to reduce hair loss due to alopecia and to promote regrowth of hair in the skin. Example inventions provide an integrated system, including patch 100, which may be placed on the skin of the user on or near the follicle. The integrated system can be activated, adjusted and used with or without the help of a medical professional, and includes hardware and software to monitor biometrics related to hair growth, and to stimulate the skin to regrow hair follicles, with the additional ability to provide a closed-loop system.

As disclosed, alopecia has several different forms: androgenetic, or male pattern baldness, but also in women; areata, a type prevalent in women; telogen effluvium; traction alopecia; alopecia universalis; and several others. In androgenetic alopecia, large terminal follicles are shed and replaced by small vellus hairs. Three areas of the scalp are affected preferentially: the temples, vertex scalp and mid frontal scalp. Within these areas the process is strictly patterned. Bitemporal hair loss starts at the anterior hairline and moves posteriorly over the scalp. Hair loss over the vertex scalp begins centrally and radiates outwards circumferentially. Over the mid frontal scalp, hair follicle miniaturization leads to a pattern of hair loss reminiscent of a Christmas tree. These three zones are not affected equally, leading to clinical variations in the pattern of hair loss, with some men balding more to the front while others bald more over the crown.

Electrical stimulation increases the rate of DNA and protein synthesis by human fibroblasts in tissue culture. Electrical stimulation increases the proliferation and alignment of smooth muscle cells and collagen in vitro in a hydrogel matrix. Electrical stimulation has also been shown in several controlled trials to improve healing of skin wounds and ulcers in animals and humans.

In the context of alopecia the effects of electrical stimulation include the following: Angiogenesis or the increase in vascularization which along with vasodilation promotes increased blood flow which will deliver increased quantity of nutrients and growth factors; natural collagen growth as the result of increased density of fibroblasts and the alignment of these fibroblasts to promote fibril formation within the collagen; fibroblasts also produce elastin and ECM (extracellular matrix); increased elasticity and suppleness of skin; Increase in the thickness of epithelium (epidermis and dermis thins as one ages); a tightening and toning of muscles due to electro muscular stimulation (“EMS”); a decrease in pore size for an overall improvement in the appearance of skin; and the creation of a bacteriostatic state.

The arrector pili muscle (“APM”) is a smooth muscle associated with each group of hair follicles, termed a follicular unit. Associated with each APM is a nerve, composed of C fibers which are controlled by the sympathetic branch of the autonomic nervous system. The detachment of the APM from the hair follicle results in follicle loss and subsequent permanent hair loss.

However, electrical stimulation technologies for reducing hair loss due to alopecia have been limited to date by the size of the stimulators and batteries and associated wires and electrodes, and by their limited functionality. They typically have one channel of stimulation with two large electrodes, with one anode and one cathode, producing an electric field and current path that penetrates several millimeters into the body. A current mode of application is a handheld device. It may include multiple dials and switches required to adjust parameters. In one mode of operation the disclosed invention applies a set charge delivered for the duration of the treatment. In another mode the disclosed invention dynamically adjusts the treatment parameters without the users' input. The disclosed invention strengthens and repairs the APM by applying an electrical charge to it via a dynamic charge profile in response to observed cellular activity. Sensors detect the response to the charge applied to the APM and hair follicle and adjust the charge for the best effects. The sensors detect the state of the hair follicle and APM via one of several sensing methods including conductivity measurements and the change in conductivity over time through its electrodes, optical estimation of hair density, or optical estimation of skin color, which indicates the level of blood perfusion in the skin.

Transcutaneous electrical stimulation enhances hair retention and regrowth through one or more of increased blood flow and vascularization, exercise and preservation of the APM associated with each group of hair follicles, regeneration of defective APMs and preservation or reinnervation of nerves of the dermal and epidermal layers of the skin. Increased collagen and elastin growth produces additional supporting structures to anchor the hair follicle and prevent hair loss. Patch 100 varies the electrical stimulation treatment parameters for optimal hair retention either through preset treatment regimens or through variable treatments based upon feedback from sensors located on the patch or from other sources including for example without limitation cellphone images, user's observations, hair professional's judgment, etc.

As disclosed below, these enhancements are generated by patch 100 using transcutaneous electrical stimulation and the voltage difference between each anode and cathode pair, which creates an electric field. Increased blood flow, vascularization and nerve growth result from the direct influence of the electric field. Exercise of the APM results from the repeated contraction of the APM when the nerve end plates on the APM or the pilomotor nerve or both are activated by voltage pulses between each anode and cathode.

The stimulation parameters including pulse shape, frequency, amplitude, adaptive pulse settings, and pulse patterns are designed to optimize the various effects of electrical stimulation. In an example, the frequency for best vascularization and increased blood flow is set at one frequency, whereas the frequency for best exercise of the APM is be set at another frequency. This difference in parameters is accommodated by the patch 100 that is fully programmable and the course of treatment will be sequenced to produce the optimal result. Other variations in electrical stimulation parameters may be accommodated by the patch 100.

Further, in example inventions, transcutaneous electrical stimulation may be used in conjunction with application of chemicals to the skin surface, accelerating the penetration of the chemical elements through the skin by iontophoresis.

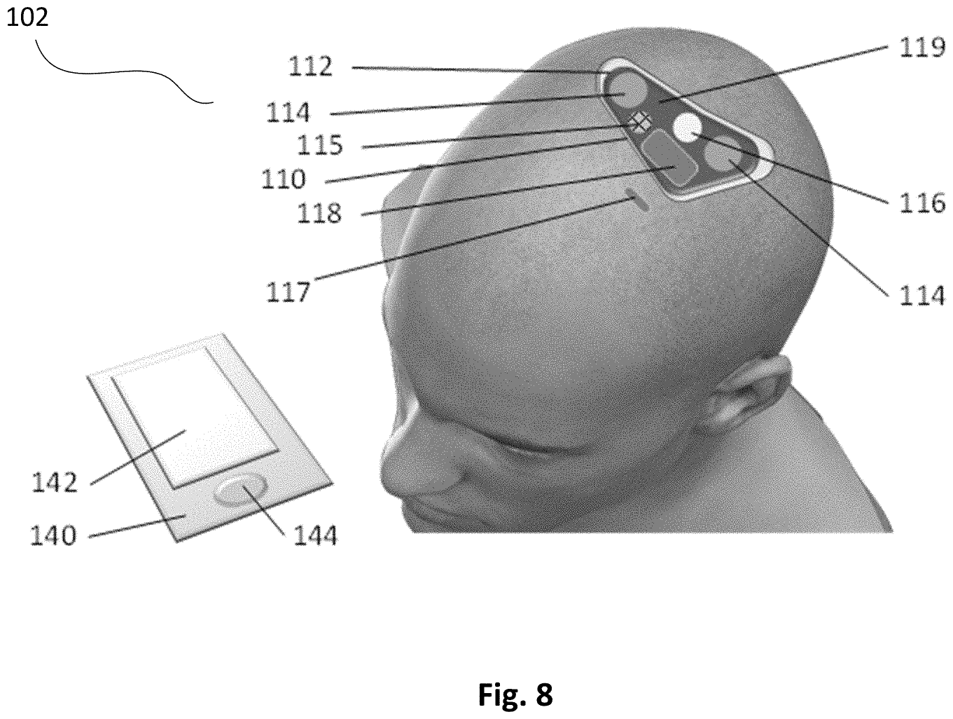

Example inventions provide electrical charge in a profile to organize cells in and around a target location to promote follicle preservation and regrowth. FIG. 8 is an illustration of components of an alopecia repair system 102 in accordance with example inventions. System 102, as shown in FIG. 8, is adapted for repairing alopecia that has occurred on the head of the user but can be placed in other areas of the user where hair regrowth or retention is desired. System 102 includes an alopecia repair patch 110, which includes a securing mechanism 112 (e.g., adhesive layer), and one or more electrode pairs 114, with each pair having a positive electrode and a negative electrode (or multiple positive electrodes and a single negative electrode as disclosed below). Patch 110 further includes a power source 116, one or more sensors 115, a processor 118 and a flexible substrate 119. System 102 further includes an optional smart controller 140 (e.g., a smart phone), with a display 142, and an acknowledgment button 144, and a tag 117. Patch 110 can be implemented by patch 100 previously described.

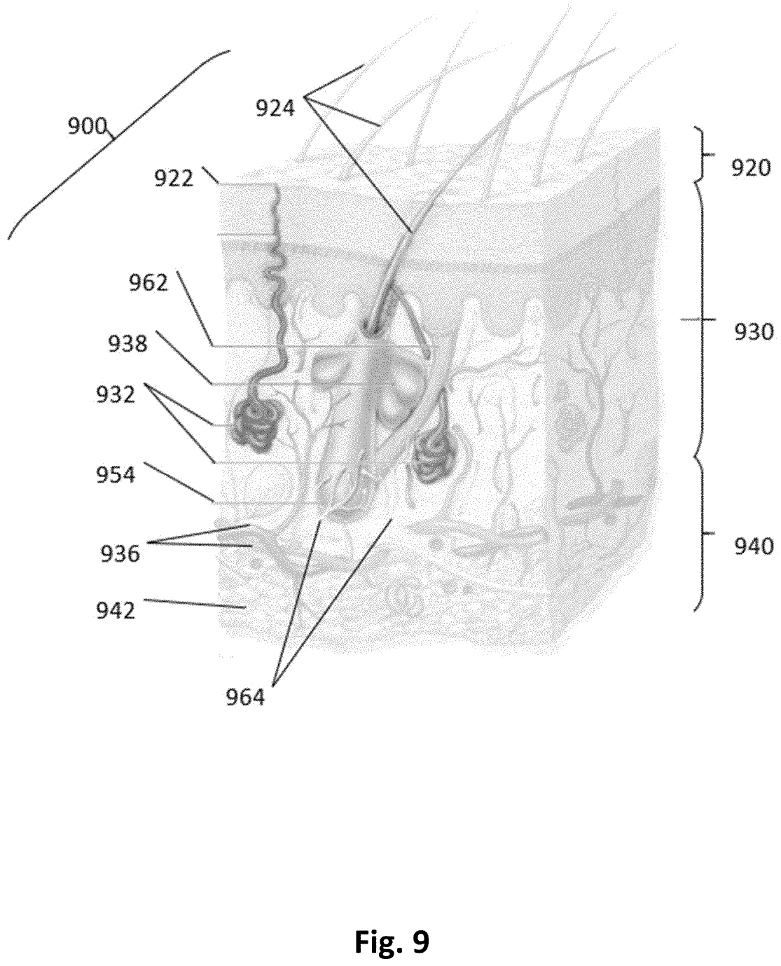

FIG. 9 illustrates a user 900 with skin 910 having epidermis 920 with pores 922 and hair shaft 924 deriving from the hair follicles. User 900 further includes dermis 930 with sweat glands 932, collagen support structures that too small to be shown in FIG. 9 and capillaries 936. Further shown are sebaceous glands 938, hypodermis 940 and fat cells 942. Further shown are a follicle area 950 with follicular units 952 and a major follicle 954 and minor follicle 954, with Arrector Pili Muscles (“APM”) 962 and Pilomotor Nerve Fibers (“PNF”) 964.

Patch 110 is designed in a shape to conform to the skin when affixed to the skin and to be electronically effective at stimulating the tissues at and near follicle area 950 in example inventions. Patch 110 may be used for alopecia treatment as well as for delivering electrical stimulation. The shape of patch 110 in examples is designed to minimize discomfort for the user 900 when affixed in the target location.

In some examples, patch 110 includes one or more electronic sensors 115 in a fixed placement on patch 110 relative to electrodes 114. Sensor 115 is used to detect the strength of the activation pulse at the target follicle area 950 through the use of tag 117 that is previously placed on or near the target follicle area 950. Tag 117 responds to the activation signal from electrodes 114 to a degree proportional to the strength of the activation signal coupled into target follicle area 950, and sends this response to patch 110. The strength of this response is then used by the user to re-orient or move patch 110 on follicle area 950 for optimum performance of the activation on target follicle area 950.

In one example, tag 117 is placed in the target follicle area 950 and is fabricated of materials such that tag 117 does not need to be removed when the follicle area treatment is concluded.

Patch 110 intervenes in the alopecia treatment processes by providing an electric field in the area of the alopecia-affected tissues. In examples, patch 110 uses one electrode pair 114 to create a lateral electric field across follicle area 950. In examples, patch 110 uses multiple positive electrodes and one or more negative electrodes to create a lateral electric field across one or more sections of follicle area 950, modifying the waveshapes or timings or both of the activation pulses from the multiple electrodes to direct the waveform energy at one or more specific points on follicle area 950.

In examples, patch 110 uses an array of electrodes surrounding follicle area 950, or an array on both sides of follicle area 950, or other configurations, modifying the waveshapes or timings or both of the activation pulses from the multiple electrodes to direct the waveform energy at one or more specific points on follicle area 950. In an example, patch 110 includes a layer of gel, such as hydrogel, between the electrode face of patch and the surface of the skin. The gel layer improves the impedance matching between the patch and the skin. Further, patch 110 facilitates that transport of drugs and other skin formulations through the dermis through pH matching, as disclosed in details below.

The individual components of alopecia repair system 102 may be connected as peer devices in a Body Area Network, passing each other signals and sharing the tasks of data recording, real-time analysis, and closed-loop monitoring of user 900. Further, alopecia repair systems 102, of different shapes or dimensions, may be used in combination to treat large or complicated follicle damaged areas. The selected alopecia repair system 102 may be adjusted during the course of repair to adapt to the changing dimension of severity of the hair loss. These multiple alopecia repair systems 102 may be connected as peer devices in a Body Area Network, with one or more controllers.

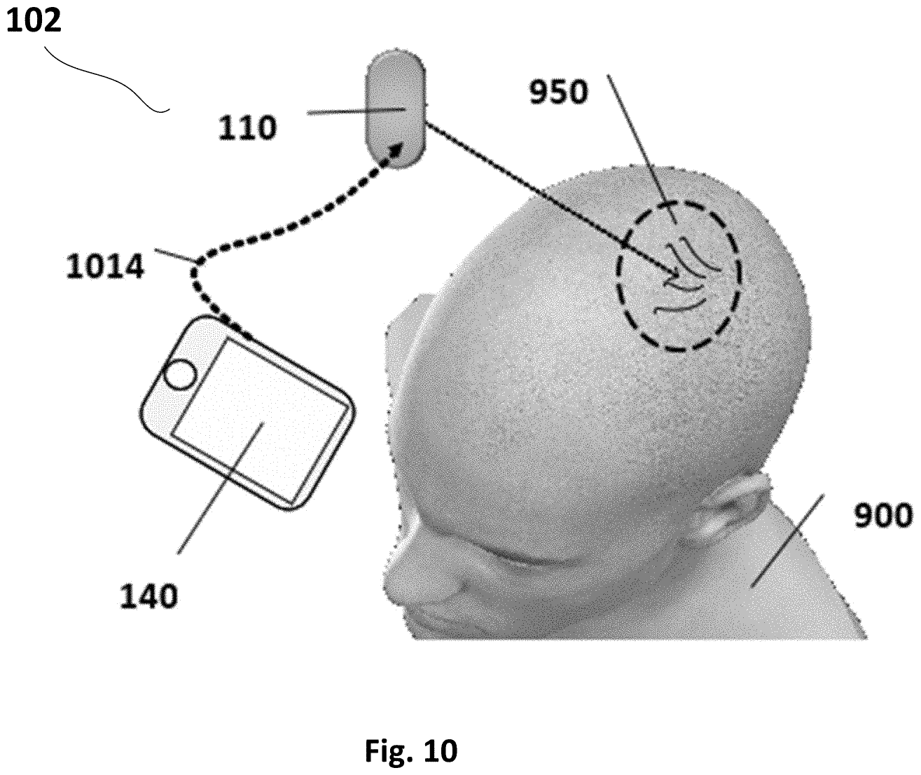

FIG. 10 illustrates another example of alopecia repair system 102. In FIG. 10, patch 110 is applied to the skin 910 at a follicle area 950 of user 900, and an optional smart controller 140 (e.g., a smartphone) is in communication with patch 110 via wireless communication link 1014. Smart controller 140 monitors biometrics from patch 910 to track the progress of hair loss reduction in the target follicle area 950. Follicle area 950 can be at any area on the body where user 900 wishes to reduce hair loss.

The communication of data and control between smart controller 140 and patch 110 may be by wireless through the use of Bluetooth Low Energy (“BLE”), Wi-Fi, or other means. Patch 110 and smart controller 140 may be powered by battery or rechargeable means.

Patch Placement

The placement of patch 110 onto follicle area 950 may be difficult for some users due to the angle of view to that part of their anatomy. For some users, aligning the device to one or more specific anatomical features, such as the center of the kneecap, may provide sufficient guidance to properly position the device. For some users, additional prompts may be required, such as in an area on the top of the head face.

In an example, a separate device, such as a smartphone or camera, is mounted on a surface or held by user 900 or held by a second person, and provides a view of the target area for patch 110 such that the user or a second person may accurately place patch 110 on the skin. In an example, a separate device, such as a smart phone or goggles, uses augmented reality features to display for user 900 certain additional images or markers in relation to one or both of the target location on the user and the real time location of patch 110 before affixing it to the user, such that these additional images or markers, or both, are used to assist the user in accurate placement at the target location.

In an example, a mark or indicator on patch 110 is used by user 900 to align the device properly on follicle area 950. As an example, a template is provided to user 900, including markings or indicators on the template to simplify positioning of the template in the prescribed position on follicle area 950. The template is used to provide a marked location for proper positioning of patch 110. The template is removed from the skin after proper placing of patch 110.

In an example, the template is disposable and used one time by user 900. In an example, the template is reusable and saved by user 900 for repeated use in aligning and positioning patch 110 on the head of user 900. As an example, patch 110 initiates a low-intensity stimulation intended to trigger a perceptible sensation in the user if and only if the device is properly positioned on the skin. This sensation may be a muscle twitch, a tingling, or similar, which provides no purpose except as a confirmation of positioning. The user may, after affixing the device to the skin and feeling no sensation, pull the device off of the skin and reposition it, repeating this process until the sensation is felt and the device is properly positioned.

Stimulation Protocol for Alopecia Repair Treatment

In examples, user 900 selects a protocol of electrical stimulation, to be applied by patch 110 to follicle area 950. The stimulation protocol may be automatically adjusted based on sensed parameters, adjusted by user 900 as the treatment of follicles progresses, or as directed by a medical professional or from data gathered by sensors on the patch or from other sources.

In examples, patch 110 stimulates follicle area 950 using a direct current (“DC”) voltage, creating an electric field from one electrode to a second electrode on patch 110, the voltage amplitude ranging from zero volts upward to 150 volts or more.

In examples, patch 110 stimulates follicle area 950 using alternating current (“AC”) voltage, creating an electric field by the frequency and amplitude, from one electrode to a second electrode on patch 110, the voltage amplitude ranging from zero volts upward to 150 volts or more, and the frequency ranging from 2 Hz to 200 Hz. In other examples, a mix of both AC and DC is used.

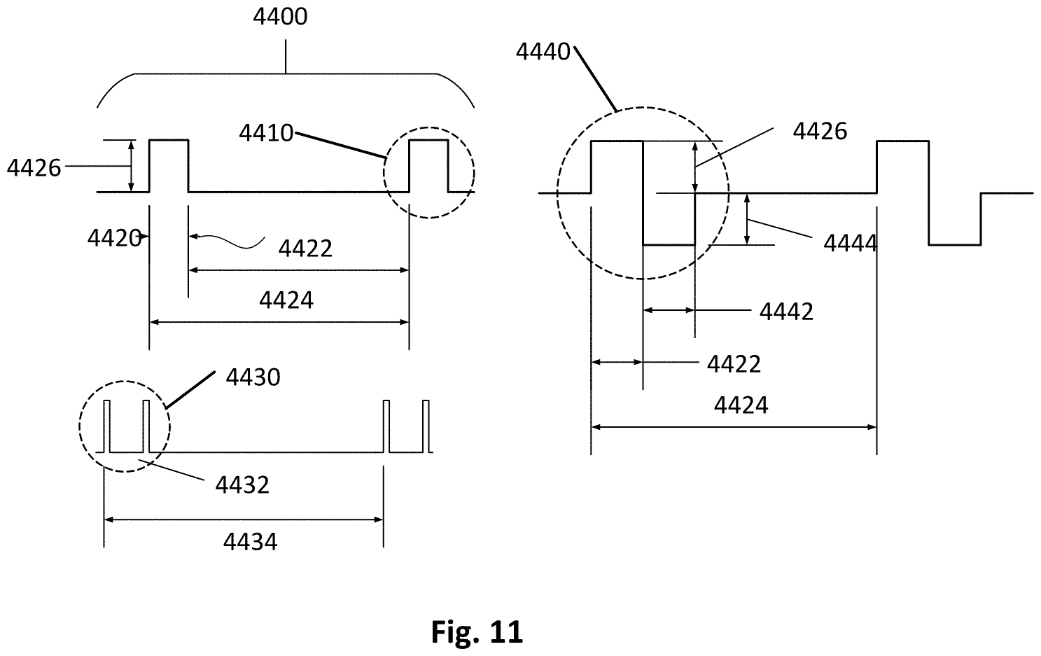

FIG. 11 illustrates example stimulation waveforms for alopecia repair in accordance with example inventions. Patch 110 stimulates follicle area 950 using a series of electrical pulses in a pattern of pulse sequence 4400 with a specific frequency, waveform, intensity and duration. Pulses 4410 may be applied at an intensity below that level which stimulates a painful sensation and below that level which wakes a user if the treatment occurs during sleep.

In examples, each pulse has a pulse high time 4420, a pulse low time 4422, a pulse period 4424, and a pulse amplitude 4426. Pulses 4410 may be monopolar pulses or bipolar pulses 4440. For monopolar pulses, the pulse period 4424 is the sum of the pulse high time 4420 and the pulse low time 4422. Bipolar pulses have a negative pulse width 4442 and a negative pulse amplitude 4444, for the purpose of balancing the DC bias of the sequence of stimulation pulses, and for the purpose of balancing for zero net energy into the tissues. The negative pulse width may differ from the pulse high time. The negative pulse amplitude may differ from the pulse amplitude. Pulse shapes are affected by the impedance coupling to the user's tissues and by the patch 3010 output impedance, internal drive strength, and other factors, such that the pulses, whether monopolar or bipolar, may not be square waves.

One or more of the pulse high time 4420, the pulse low time 4422, the pulse period 4424, and the pulse amplitude 4426 may be adjusted. For bipolar pulses 4440, the negative pulse width 4442 and negative pulse amplitude 4444 may be adjusted from one user to another user, or from one application of a device to another on the same user. The pulse pattern may be adjusted during the course of a treatment.

Pulses may be output in bursts 4430. Each burst has two or more pulses 4410, or bipolar pulses 4440. Each burst has a burst pulse count 4432 and a burst period 4434. One or both of the burst pulse count and the burst period are adjustable for each user, or from one application of a device to another on the same user. The pulse frequency is the inverse of the pulse period. The burst frequency is the inverse of the burst period. Pulses or bursts may be adjusted by each user each time a patch 110 is applied, since effective intensity may be different according to skin condition, dampness, dryness, weight change, specific location of placement and other factors. In this manner, the electrical pattern of stimulation pulses is adjusted for each application/treatment.

In an example, the polarity of the electrical stimulation may be reversed during the course of an alopecia treatment. In examples, the pulses within one burst may all be of equal width. In examples, the pulses within one burst may be of varying widths, the width adjusted to optimize the stimulation for effectiveness.

In an example, the applied frequency of the stimulation pulses 4410 is in the range of 2 Hz to 150 Hz, and the current applied is up to 10 milliamps, or up to 100 milliamps. In an example, pulses 4410 singly or in bursts 4430 have pulse high times 4420 in the range of 100 to 500 microseconds, and pulse low times 4422 in the range of 100 to 500 microseconds, and with burst frequency and pulse frequency for single pulses in the range of 2 Hz to 50 Hz, and the current applied is up to 40 milliamps.

In examples, the pulses within one burst may be evenly spaced and/or are all of equal width. In examples, the pulses within one burst may be unevenly spaced and/or have varying widths, with the width adjusted to optimize the stimulation for effectiveness. In examples, the pulses within one burst may have consistent amplitude. In examples, the pulses within one burst may have unequal amplitudes.

In an example, one or more of pulse rise time, pulse fall time, pulse overshoot, and pulse undershoot are adjusted by one or both of patch 110 and smart controller 140. Changes in pulse shape, including one or more of rise time, fall time, overshoot and undershoot, allow the patch to optimize use of power during the application of a treatment protocol. Optimizing the power used in a treatment allows a patch with a given design to apply more stimulation when compared to a patch without the means to optimize power delivery. Pulse shape is limited by one or both of patch and smart controller such that delivered energy, rate of energy delivery, magnitude of currents and/or voltages all meet the requirements for effective tissue stimulation at the applied position.

In an example, smart controller 140 adjusts the intensity of applied pulses, or the duration of application, or both, using data exchanged with patch 110 and its processor 118. The exchanged data includes data from the monitoring device or devices, described below included in alopecia repair system 102.

In an example, one or both of patch 110 and smart controller 140 adjusts the intensity or the duration of the applied pulses, or both. In an example, user 900 adjusts the intensity and the duration of the applied pulses, or both. All adjustments may be limited to preset ranges.

In an example, one or both of patch 110 and the smart controller 140 operate to select one of a variety of hardware configurations, each hardware configuration on the patch specified to limit one or more of pulse rise time, pulse fall time, pulse overshoot, and pulse undershoot. One example uses a bank of capacitors, switched into the pulse application circuit under control of the patch, to optimize the load and its effect on the driven pulse voltage and current. A second example uses a bank of inductive loads. A third example uses a bank of resistive loads.

Alopecia Repair Monitoring

Example inventions monitor follicle area 950 at the beginning of the treatment and/or during the treatment to optimized the repair process and to monitor the progress of the repair. The analysis is performed on data collection measurements from one or both of smart controller 140 and patch 110 (via sensors 115) and may be performed by patch 110, smart controller 140, or by processing in a remote server, in the cloud, or on a computer separate from smart controller 140 but local to the user, such as a personal computer. The system analyzes this data and determines the most effective times to start and end each treatment protocol to optimize follicle preservation and growth.

In an example, follicle repair system 102 measures the impedance at the surface of the skin at follicle area 950, which is affected by moisture levels and other factors. In an example, follicle repair system 102 measures the pH at the surface of the skin at follicle area 950. In an example, follicle repair system 102 measures the color of tissues at follicle area 950 using one or more photo sensors, in visible or infrared wavelengths.

Other sensors include an electromyography sensor (“EMG”) in which the EMG signals change with the alopecia repair progress and sensors for measuring compounds emitted from the skin, such as VOCs, sweat, and other compounds (e.g., a semiconductor gas detection chip).

In other examples, patch 110 includes a clear area in approximately the middle of the patch to allow a visual observation of the follicle repair area and the progress of the repair.

In an example, follicle repair system 102 collects time-based records of a user's tissue. These records are entered into a database of anonymized tissue information from large populations of users of other follicle repair systems 102.

In an example, follicle repair system 102 uses artificial intelligence (“AI”) techniques such as pattern recognition and correlation analysis to correlate real-time data recordings of the user with larger population databases to produce comparative or predictive analyses. In an example, machine-learning algorithms are employed to build up the user's follicle preservation or growth history and provide specific predictors of follicle density or to provide adaptive treatment regimes. Example inventions use AI to generate a predictive skin model using, for example, a trained neural network. For example, inputs to the model can include ingredients/compounds/formulations applied to the skin or included in the patch (e.g., minoxidil, glycerin, aloe vera, Vitamin A, etc.), electrical stimulation parameters, the environment (e.g., temperature, humidity, etc.), skin conditions (e.g., pH level), skin microbiome/flora, etc. Outputs of the model can include the pH of the formulation (i.e., after applying the electoral stimulation), a predicted depth of penetration of the formulation, alerts for skin conditions/allergies, etc. Further, example inventions can be implemented as part of an augmented reality system in which treatment is adjusted based on feedback from the AR system and/or user perception.

In an example, the time-based records of tissues are supplemented with data entered manually by one or more observers of the user's tissues. The data recorded in the time-based database is sent to the cloud through a local network, such as a home mesh network, or directly over the Internet.

The convenient and comfortable use of follicle repair system 102 by user 900 allows the system to collect data over a longer period of time without undue interference with follicle growth compared to conventional approaches to follicle growth.

Electrode Arrangements

In examples, patch 110 can use multiple positive electrodes in an array or matrix and also include multiple negative electrodes. Each positive electrode creates an electric field with the negative electrode nearest to it, such that the charge flows from one electrode to the other. Each positive electrode's field is not affected by other negative or positive electrodes, as these other electrodes are electrically distant from the positive electrode and the negative electrode. However, this set of electrodes may complicate the physical and electrical layout of the patch.

Therefore, in example inventions, a set of positive electrodes instead shares only one common negative electrode, such that the return current path back to the stimulating circuit is through the one negative electrode. This common negative electrode is larger than individual negative electrodes for each positive electrode when considering the two approaches on a fixed patch area. By making the common negative electrode larger, its impedance can be lower to the skin, its fringe area is minimized such that uncomfortable stimulation sensations are minimized when compared to current paths through small electrodes, and leakage currents are minimized because the single, larger negative electrode may be more easily isolated from circuitry than a multiplicity of negative electrodes.

The set of positive electrodes may be connected to the stimulating circuit one at a time or more than one at a time, using low-impedance switches between the shared voltage generating stimulation circuit and the individual electrodes. The controller controls the switches, such that only the desired positive electrode or electrodes are connected at one time.

The patch may use one positive electrode and a set of negative electrodes. The positive electrode is driven by the voltage for stimulation, using one circuit and working through the lower impedance of the large, common positive electrode in its contact with the skin. The negative electrodes may be a common ground, and connected to each other by conductive paths on the patch and further back to the stimulating circuit to complete the current loop. Alternatively, each negative electrode may be connected to the common ground through a low-impedance switch, the switches being under control of the controller, such that only the desired negative electrode or electrodes are connected to ground at one time, thereby limiting the return current path.

The set of positive electrodes driven by a stimulation voltage may have individually adjusted stimulation voltages such that, when connected and stimulating the skin, the combined stimulation from multiple positive electrodes is more effective than identical stimulation waveforms from all positive electrodes. The currents from each of the positive electrodes passes through the common negative electrode and back to the stimulating circuit. Individual stimulating circuits create individual stimulating waveforms which have specific setups under control of the controller. The controller may adjust the amplitude, phase, pulse width, and frequency of each circuit to create a combination of stimulation through multiple positive electrodes

Sensing

As discussed, patch 110 may include one or more sensors 115. The sensors can be used to sense the state of the follicle area when treatment is initiated and during a treatment. A treatment session can be automatically initiated, modified or ended based on the sensing. The sensors can allow a baseline to be established before treatment, and the sensors can be then used as treatment progresses to detect changes. Further, the feedback provided by the sensors during the treatment can be used to adjust treatment parameters to improve the outcome. For example, the pulse frequency can be adjusted. Other changes in examples in response to sensing include the amount of current/voltage/charge delivered, the rate of treatment (e.g., frequency of pulses, the treatment course such as on 5 min., off 5 min., on 5 min . . . etc.), feedback to the user regarding the progress of alopecia repair, possibly to take advantage of placebo effects, and the need to switch to a different patch either of the same type or another type that may have different compounds incorporated into its hydrogel/adhesive.

In general, when patch 110 is applied to the skin and then uses sensors to detect when to stimulate, or how to adjust the stimulation, it uses sensing circuits that are separate from the circuits used for electrical stimulation. When the detection mechanism involves electrical signal sensing, the sensors use electrodes on the skin-facing surface of the patch. The controller monitors certain conditions through electrical signal sensing, then turns electrical stimulation on or off according to the treatment regime associated with the sensed condition. Patches use separate sensing electrodes and stimulation electrodes since each has different requirements.

However, separate sensing and stimulating electrodes increases the size of the patch and may require accurate placement of the patch. In contrast, in some examples, patch 110 uses the same set of electrodes for sensing as for stimulating. The connections to the controller are shared between sensing and stimulating functions, or the connection to each electrode is routed to unique controller pins with a low-impedance switch. The state of the switch is controlled by the controller, multiplexing sensing and stimulating functions.

Sensing requires a relatively high-impedance path from the skin surface to the analog-to-digital converter (“ADC”) circuit. The ADC may be a discrete component, passing a digital signal on to the controller, or the ADC may be integrated in the controller on one or more pins. High-impedance is required to generate a voltage proportional to the biometric, such as in EMG, the voltage having a range large enough to discriminate a wide set of values when digitized.

Stimulation requires a relatively low-impedance path to the skin surface, such that the driving circuit can overcome the impedance and drive energy into the tissue for treatment.

The two competing requirements may be combined through the use of a low-impedance or matched-impedance switch. The switch routes the signal captured at the electrode to either the sensing pin or the driving pin. For example, a single pin on the controller may be programmable to low- or high-impedance, and be able to both sense and drive into its load.

In another example, a small part of a larger stimulating electrode may be electrically isolated in the layout such that the small part may work as a sensing electrode when connected to the sensing circuit, and yet may work as part of the overall stimulating electrode when connected to the stimulating circuit. The isolation may be through two switches, one with low impedance for the sensing function, the other with impedance matching the overall impedance of the larger electrode. This latter aspect helps to minimize reflections and aberrations in the stimulating waveform when the stimulating circuit drives both the larger electrode area and the connected smaller area.

In another example, a patch uses a set of small electrodes to stimulate the skin. The overall impedance of the stimulating patches in combination is low, to optimize the effectiveness of the stimulation. The impedance of each individual small electrode is higher, such that it is effectively used in a sensing circuit.

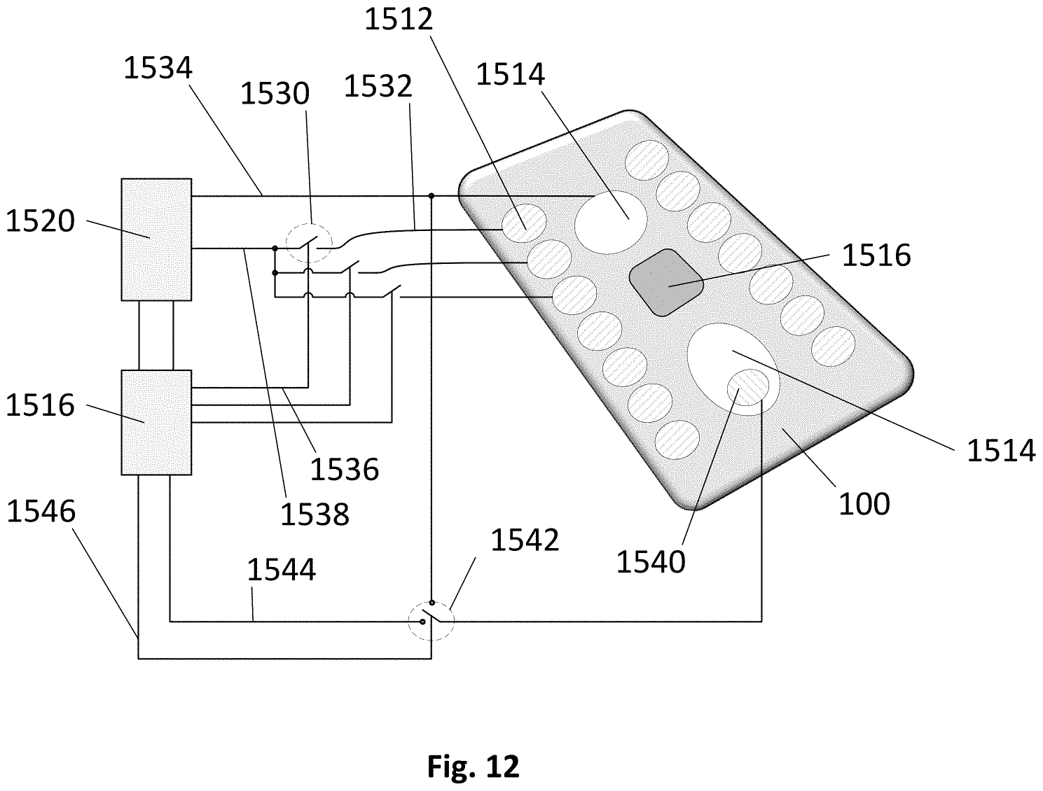

FIG. 12 illustrates patch 110 with multiple electrodes that are adapted to provide both stimulation and sensing in accordance with example inventions. Patch 110 includes a set of 14 positive electrodes 1512; and a set of 2 negative electrodes 1514. Patch 110 further includes a processor 1516 shown in a physical view and schematic view. Patch 110 further includes a stimulation voltage circuit 1520, a set of stimulation switches 1530 with a stimulation voltage wire 1532 and a return current wire 1534. Patch further includes a stimulation switch control wire 1536, and a sensor electrode 1540 with a sensing wire 1544, a sensing mode switch 1542, and a sensing mode wire 1546. FIG. 12 illustrates only 3 of the necessary 14 stimulation switches and associated wires that would be included in this example invention.

In operation, patch 110 selects one or more of positive electrodes 1512, connecting each to stimulation voltage circuit 1520 with the corresponding stimulation switch 1530. The stimulation voltage passes from stimulation voltage circuit 1520 to all of the selected positive electrodes 1512, then as a field to negative electrodes 1514, and back to stimulation voltage circuit 1520. In example inventions, patch 110 selects the subset of the available positive electrodes 1512 to optimize the stimulation of the underlying tissue. The selection is adjusted in the software or firmware of processor 1516 according to the positioning of patch 110 on or near the target area.

Further, in example inventions, patch 110 selects the one or more sensor electrodes 1540 by activating sensing mode switch 1542 to connect the sensor to processor 1516. Processor 1516 uses one or more of hardware or software or firmware to analyze the measurement procured from sensor electrode 1540, using the analyzed measurement to inform the selection of positive electrodes 1512. Patch 110 changes the mode of sensing mode switch 1542 to connect sensor electrode 1540, or to return current wire 1534 when the electrode is used during a stimulation.

Data Manager

In examples, patch 110 includes a data manager implemented by control unit/processor 1002, which has primary responsibility for the storage and movement of data to and from the communications controller, sensors, actuators, and a master control program. The data manager has the capability to analyze and correlate any of the data under its control. It provides logic to select and activate nerves or treat follicle damaged tissues. Examples of such operations upon the data include: statistical analysis and trend identification; machine learning algorithms; signature analysis and pattern recognition, correlations among the data within a data warehouse, a therapy library, tissue models, electrode placement models, and other operations. There are several components to the data that is under its control as disclosed below.

The data warehouse is where incoming data is stored; examples of this data can be real-time measurements from the sensors, data streams from the Internet, or control and instructional data from various sources. The data manager will analyze data that is held in the data warehouse and cause actions, including the export of data, under master control program control. Certain decision making processes implemented by the data manager will identify data patterns both in time, frequency, and spatial domains and store them as signatures for reference by other programs. Techniques such as EMG, or multi-electrode EMG, gather a large amount of data that is the sum of hundreds to thousands of individual motor units and the typical procedure is to perform complex decomposition analysis on the total signal to tease out individual motor units and their behavior. The data manager will perform big data analysis over the total signal and recognize patterns that relate to specific actions or even individual nerves, tissues or motor units. This analysis can be performed over data gathered in time from an individual, or over a population of patch users.

The therapy library contains various control regimens for patch 110. Regimens specify the parameters and patterns of pulses to be applied by patch 110. The width and amplitude of individual pulses may be specified to stimulate follicle related tissues. There are preset regimens that may be loaded from the cloud or 3rd party apps. The regimens may be static read-only as well as adaptive with read-write capabilities so they can be modified in real-time responding to control signals or feedback signals or software updates.

The tissue models are specific to the electrical properties of particular body locations where patch 110 may be placed. Electric fields for production of action potentials or the stimulation of tissues will be affected by the different electrical properties of the various tissues that they encounter. The tissue models are combined with regimens from the therapy library and the electrode placement models to produce desired actions. MRI, ultrasound or other imaging or measurement of tissue of a body or particular part of a body may develop tissue models. This may be accomplished for a particular user and/or based upon a body norm. One such example of a desired action is the use of a tissue model together with a particular electrode placement model to determine how to focus the electric field from electrodes on the surface of the body on a specific deep location corresponding to the nerve or tissue in order to stimulate the nerves or tissue to selectively enable alopecia repair or reduce incontinence of urine, or to treat constipation and FI. Other examples of desired actions may occur when a tissue model in combination with regimens from the therapy library and electrode placement models produce an electric field that stimulates targeted nerves or tissues.

Electrode placement models specify electrode configurations that patch 110 may apply and activate in particular locations of the body such as the sole of the feet versus the torso. For example, patch 110 may have multiple electrodes and the electrode placement model specifies where these electrodes should be placed on the body and which of these electrodes should be active in order to stimulate a specific structure selectively without stimulating other structures, or to focus an electric field on a deep structure. An example of an electrode configuration is a 4 by 4 set of electrodes within a larger array of multiple electrodes, such as an 8 by 8 array. This 4 by 4 set of electrodes may be specified anywhere within the larger array such as the upper right corner of the 8 by 8 array. Other examples of electrode configurations may be circular electrodes that may even include concentric circular electrodes. Patch 110 may contain a wide range of multiple electrodes of which the electrode placement models will specify which subset will be activated. Electrode placement models complement the regimens in the therapy library and the tissue models and are used together with these other data components to control the electric fields and their interactions with nerves, muscles, tissues and other organs. Other examples may include patch 110 having merely one or two electrodes, such as but not limited to those utilizing a closed circuit.

Stack-Up of the Patch

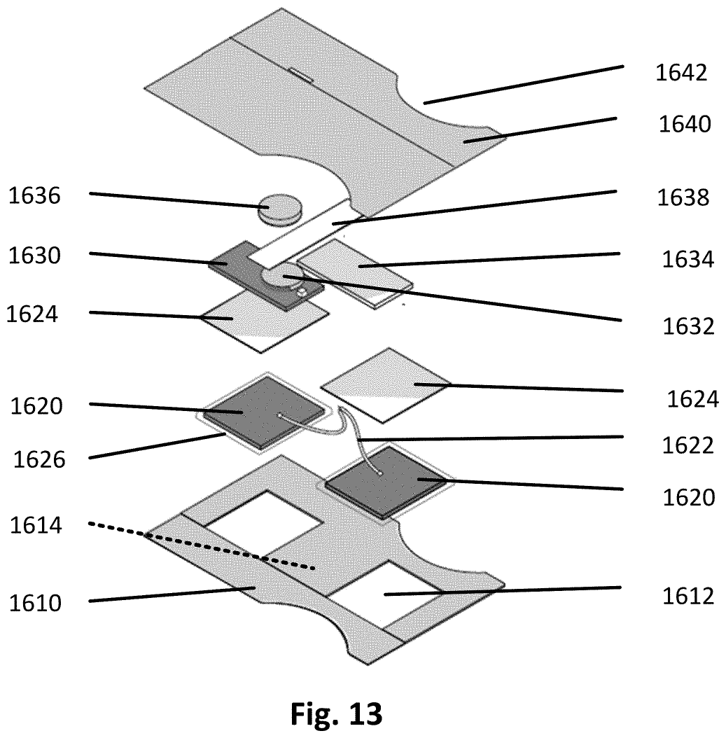

FIG. 13 illustrates a stack-up view of patch 110 in accordance with example inventions. A bottom layer 1610 is a fabric tape with adhesive on the skin-facing side. A hole 1612 is cut into the bottom layer for each of the electrodes 1620. A removable paper 1614 adheres to the adhesive on the skin-facing side of bottom layer 1610. Two or more electrodes 1620 are coupled by a wire 1622 to a printed circuit board assembly (“PCBA”) 1630.

Electrodes 1620 are covered with a polyimide tape A 1624 to prevent short circuits from electrodes 1620 to PCBA 1630 and to prevent movement of electrodes 1630 within the layers of the assembly. Each electrode 1630 is coated on the skin-facing surface with hydrogel 1626. Each electrode 1620 has a release layer covering hydrogel 1626. A battery clip 1632 is attached to PCBA 1630. A battery 1636 is inserted into battery clip 1632. A battery pull-tab 1638 is inserted into battery clip 1632. PCBA 1630 is wrapped in polyimide tape B 1634 to restrict access by the user to the electronics. A top layer 1640 of fabric tape with adhesive on the PCBA-facing side is stacked on top to complete the assembly.

Hydrogel Adaptation

Variations in the viscosity and composition of hydrogel 1626 leads to variation in the migration of the substance from its original area on each electrode to a wider area, possibly touching the skin outside the dimensions of patch 110. As the hydrogel migrates, its electrical performance changes. The circuitry on PCBA 1630 measures the voltage applied to the skin in real-time during the course of each treatment. The adaptive circuit calculates the charge delivered to the skin, which is a function of many parameters, including the conductivity of hydrogel 1626. Therefore, the performance of patch 110 is maintained while the hydrogel portion of the device changes its performance. The adaptive circuit adjusts the delivery of charge to also account for all changes in body and skin conductivity, perspiration and patch contact.

As the performance of the hydrogel 1626 decreases with time, the adaptive circuit and the firmware in PCBA 1630 records the expected life of the specific patch while it is powered on and on the skin of the user. When patch 110 determines that the device's lifetime is near an end, the firmware signals to the fob or smart controller, such that the user receives an indication that this patch has reached its limit.

Crimped Connection from Electrode to PCBA

Each electrode 1620 is coated with hydrogel 1626 when the electrode is manufactured. In some examples, soldering, when electrodes 1620 are manufactured, connects a wire 1622 to both the electrode and the PCBA 1630 in a permanent fashion. The electrode-plus-wire-plus-PCBA assemblies are each enclosed in an airtight bag until they are subsequently assembled with the tapes and adhesive layers to form a complete patch 110. Due to the complex nature of these assembly steps, the hydrogel on the electrodes may be exposed to air and humidity for a period of time, which affects the life expectancy of the hydrogel.

In an example, electrodes 1620 are coated with hydrogel 1626 but no wire is attached at that stage. Instead, a small clip is soldered to each electrode which does not affect the hydrogel nor attach the electrode to any larger assembly which would require longer time in the assembly line. These coated electrodes are each encased in an airtight bag with a heat seal or other means. The hydrogel does not degrade during the time that the coated electrode is inside the sealed bag.

In an example, wire 1622 is inserted into the small clip which had previously been soldered to electrode 1620, this connection being stronger and less prone to defect than the soldering or attachment of the wire strands directly to electrode 1620. The clip and the wire do not affect hydrogel 1626. Each coated electrode 1620, with its clip and attached wire, is encased in an airtight bag with a heat seal or other means. Hydrogel 1626 does not degrade during the time that the coated electrode is inside the sealed bag. The coated electrodes 1620 are removed from their airtight bags only immediately before they are connected to PCBA 1630.

An additional benefit of separating the coated electrodes 1620 from PCBA 1630 as two different subassemblies until put into a completed patch 110 is that coated electrodes found to be defective or expired from too lengthy time on the shelf may be discarded without the expense of discarding an already-attached PCBA. The more expensive PCBAs have a shelf life independent of the shelf life of the coated electrodes. These two subassemblies' inventories may be stocked, inspected and managed independently. This reduces the overall cost of manufacture of patches 110 devices without affecting their performance.

Die Cut Fabric Tape

In some examples, bottom layer 1610 is placed as a layer over electrodes 1620 using a solid layer of fabric tape. The overall thickness of patch 110 is therefore partly determined by the thickness of the fabric tape over electrodes 1620. Further, in order to place electrodes 1620 on the layer of fabric tape securely, the paper cover on the fabric tape must be pulled back to expose the adhesive coating. This results in a degradation of the adhesive properties of the tape.

In examples of patch 110, bottom layer 1610 fabric tape is cut to create holes 1612 for each of electrodes 1620, according to the defined sizes of those components. Each electrode 1620 is placed in the corresponding hole, without the added thickness of a fabric tape layer on top. Since no paper cover needs to be pulled back to mount electrodes 1620 to the fabric tape, the adhesive of the fabric tape is not affected. The holes may be cut with a die in order to create accurate edges, without tears or fibers, which may interfere with electrodes 1620.

Battery and Battery Tab

Patch 110 includes battery 1636, which is enclosed by battery clip 1632, assembled onto PCBA 1630. During manufacturing, battery 1636 is inserted into battery clip 1632 to secure it from dropping out. In addition to the battery itself, battery pull tab 1638 is placed between one contact of battery 1636 and the corresponding contact in battery clip 1632. Battery pull tab 1638 prevents electrical connection between battery 1636 and battery clip 1632 at that contact until battery pull tab 1638 is removed. When in place, there is an open circuit such that patch 110 is not activated and does not consume power until battery pull-tab 1638 is removed.

In some examples, battery pull-tab 1638 is designed to be removed by pulling it out in the direction opposite that in which battery 1636 was inserted into battery clip 1632. This pulling action may lead to movement of the battery itself since it experiences a pulling force toward the open side of battery clip 1632. This battery movement may cause patch 110 to cease operating or to never activate.

In one example, battery pull-tab 1638 and battery clip 1632 are designed so that battery pull tab 1638 is pulled out in the same direction as battery 1636 was pushed into battery clip 1632. Therefore, the force pulling battery pull-tab 1638 out of patch 110 serves only to make battery 1636 more secure in its battery clip 1632. This reduces the chance of inadvertent movement of battery 1636 and the effect on activation or operation of patch 110.

Electrode Release Film

Each of electrodes 1620 in the assembled patch 110 is covered with a Polyethylene Terephthalate (“PET”) silicon covered release film 1626. The release film is pulled away by the user when patch 110 is affixed to the skin. In some examples, the PET silicon covered release film 1626 is transparent. This may lead to instances of confusion on the part of the user when the user may not be able to determine if the tape has been removed or not. Affixing patch 110 to the skin with any of electrodes 1620 still covered with tape will cause patch 110 to be ineffective. This ineffectiveness may not be noticed until the first treatment with patch 110.

In examples, PET silicon covered release film 1626 covering electrodes 1620 is selected in a color conspicuous to the user, such that the user will readily determine if the tape has been removed or not.

Examples use circuitry and firmware to stimulate the electrode circuit with a brief, low energy pulse or pulse sequence when patch 110 is initially activated. If patch 110 is activated before it is affixed to the skin, the electrode readiness test will fail. In such a case, the electrode readiness test is repeated, again and again according to timers in the firmware or hardware, until either the timers have all expired or the test passes. The test passes when patch 110 is found to exhibit a circuit performance appropriate to its design. The test fails when patch 110 is not properly prepared, such as not removing the electrode films, or is not yet applied to the skin when the timers have all expired. When the electrode readiness test fails, patch 110 signals to the fob or the smart controller, which in turn informs the user. The electrode readiness test is implemented in a manner which may be undetectable by the user, and to minimize the test's use of battery power.

Removable Paper

In some examples, a removable paper 1614 covers the adhesive side of bottom layer 1610. Removable paper 1614 may be in multiple sections, each to be pulled away by the user when affixing patch 110 to the skin. These removable papers may be in addition to the piece of PET film 1626 covering each electrode 1620. Therefore, the user must remove all of these pieces to expose a complete, adhesive surface to affix to the skin in examples.

In examples, bottom layer 1610 is one complete piece, with one removable paper 1614. The user removes all of the removable paper in one motion. In examples, bottom layer 1610 is two or more pieces, with two or more removable papers 1614. The user removes all of the removable papers. In examples, the single removable paper 1614 is designed with a pull-tab, so that the user pulls the removable paper off of the bottom layer in a direction at right angle to the long axis of patch 110. This motion reduces the forces experienced by the assembled internal components of patch 110.

In examples, removable paper 1614 covers bottom layer 1610 and covers all of the PET film sections 1626. An adhesive attaches the removable paper top surface to the polyimide tape A skin-facing surface, such that the user pulls the removable paper away from the bottom layer and in one motion removes the PET film pieces from electrodes 1620.

Patch 110 can also be made more comfortable by the addition of material between the top layer and the bottom layer, such as cushioning material that can cushion the electrodes and electronic components. The cushioning material may be disposed subjacent to the bottom layer and superjacent to the top layer, in at least a portion of patch 110. A cushioning material may include cellulosic fibers (e.g., wood pulp fibers), other natural fibers, synthetic fibers, woven or nonwoven sheets, scrim netting or other stabilizing structures, superabsorbent material, foams, binder materials, or the like, as well as combinations thereof.

Hydrogel Overlaps Electrode Edges

In some examples, each electrode 1620 is covered with hydrogel 1626, which conforms to the size of the electrode 1620, such that the edge of electrode 1620 is exposed to the user's skin when patch 110 is applied to the skin. This edge may abrade or cut the user's skin during the time when patch 110 is affixed to the skin.

In some examples, hydrogel 1626 is dimensioned so as to overlap the edges of electrode 1620. Hydrogel 1626 is placed over electrode 1620 with the accuracies of placement used in manufacturing, such that the edges of electrode 1620 is always covered with hydrogel 1626. This keeps the edge electrode 1620 from touching the user's skin. The risk of electrodes 1620 from abrading or cutting the user's skin is therefore eliminated.

Iontophoresis and the Alopecia Repair System

Iontophoresis is the transfer of molecular compounds through the epidermis into the deeper tissue using the application of electric fields to the skin. The voltage gradient created by the electric field increases the permeability of the skin, allowing the transfer of molecules larger than those which can permeate the skin without the applied field. Molecules are transported across the stratum corneum and other epidermal layers by electrophoresis and electroosmosis.

Patch 110 may be applied to the skin after one or more compounds have been applied to the surface of the skin or the compounds may be incorporated into the patch. In the case that patch 110 does not incorporate the one or more compounds, patch 110 can be placed directly over the area of the compound application such that patch 110 applies an electric field to increase the permeability of the skin to the one or more applied compounds and medicines through iontophoresis. By driving these compounds into the deeper layers of tissue than is possible without patch 110, the compounds are more effective at alleviating alopecia and improving skin health.

In an example, the one or more compounds for iontophoretic transfer are incorporated into the hydrogel or adhesive layer, or both, as part of patch 110. These compounds are too heavy to penetrate without the iontophoretic effect of patch 110.

In an example, precursors of compounds useful to follicle treatment are incorporated into the hydrogel or adhesive, or both, as part of patch 110. These precursors form larger compounds which are too heavy to travel through the skin after transferring into the epidermis or hypodermis. In an example, patch 110 may be used in combination with a separate dressing or bandage to hold the system in place on the body.

Further, in example inventions, patch 110 is used to modify the pH value of the dermis it is affixed to and/or the pH value of compounds or medicine (e.g., minoxidil, finasteride, etc.) to be applied to the dermis in conjunction with alopecia repair. In example inventions, patch 110 attempts to equalize the pH level of the skin and the compounds, which facilitates the transfer of the compounds through the dermis.

For example, the optimal pH value of skin on most of a user's head and body lies between 4.7 and 5.75. A pH of 7 (i.e., that of pure water) is considered neutral. Anything below that is acidic and above it alkaline, so the skin's natural pH is mildly acidic. A skin's ideal pH is 5.5, which is slightly acidic and is when the skin is generally optimized.

In contrast, the pH level of various compounds that may be applied to the skin in order to enhance alopecia repair is as follows: cleansers: pH 4.5-7; toners: pH 5-7; sunscreens: pH 5-7.5; AHA and BHA exfoliants: pH 3.2-3.9, with any reading between 3 and 4 considered most effective. Further, the key ingredients present in most cosmetics include water, emulsifiers, preservatives, thickeners, moisturizers, colors and fragrances, which are mostly large molecule formulations, so therefore difficult to pass through the dermis. Ingredients can be naturally occurring or artificial, but any potential impact on health depends mainly on the chemical compounds they are made of. Ingredients can either enhance the alopecia repair or enhance skin health in conjunction with the alopecia repair. The ingredients include, but are not limited to, Vitamin C (Ascorbic Acid), Hyaluronic Acid, Collagen, Vitamin A (Retinoids), Broad-Spectrum Sunscreen, Lactic Acid, Ceramides, and others.

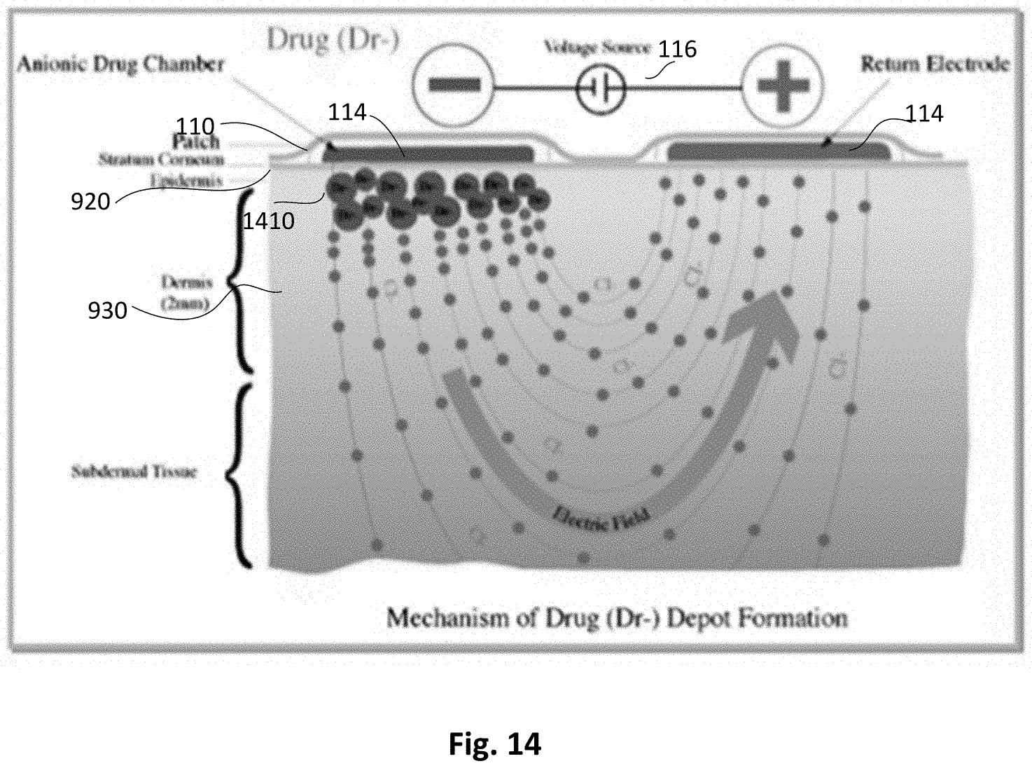

FIG. 14 illustrates a sectional view of patch 110 affixed to dermis 930 in accordance to example inventions. FIG. 14 illustrates how a compound/medicine/solution 1410 is pulled through epidermis 920 via iontophoresis as a result of an electric field generated by electrode pair 114 and voltage source 116.

In examples, patch 110 approximates a match of the pH of epidermis 920 with the pH of compound 1410 (which facilitates the iontophoresis) by controlling the respective pH levels. There is a relation between the pH and the electrical current flow (i.e., the conductivity) of a solution, and there are other factors that affect the conductivity.

When an electrical current passes through a wire, what actually moves and carries the current are electrons. However, in solutions, the current is carried by IONS: positively charged CATIONS⁺ such as H⁺, Na⁺, Mg⁺, etc., and negative ANIONS [−] such as OH⁻, Cl⁻, Acetate⁻, etc. The conductivity of a solution depends on the concentration of ALL the ions present, the greater their concentrations, the greater the conductivity. These ions all have the electrical unit charges shown by their symbols, but they move at different velocities (i.e., mobilities) through the solution, so they contribute differently to the conductivity. Of the common ions, the most mobile CATION is the Hydrogen ion (H⁺) with a value of 350 units, and the most mobile ANION is the Hydroxyl ion (OH⁻), 199 units. The other common ions have values ranging between 40 and 80 units. Therefore, a strongly acidic (or strongly basic) solution will have high conductivity. Since the pH is a measure of the concentration of the Hydrogen (and the Hydroxyl) ions, for an acidic solution, the lower the pH (i.e., the higher the H⁺ concentration), the greater the conductivity will be, because conductivity is the sum of the contribution of all the ions present in the solution. Calculations of current related pH are only valid if all the ions are taken into account. Water ions (H⁺) are only a part of the calculation.

Example inventions use sensors 115 on patch 110 to measure the pH of the skin and/or the formulation, and then adjusts the stimulation parameters to drive the pH to a desired reading through a closed feedback loop. The adjusted stimulation parameters may be voltage, current, pulse shapes, frequency, pulse patterns, etc. In example inventions, sensors 115 include an ion-sensitive field-effect transistor (“ISFET”) sensor that measures the pH levels. Feedback can also be obtained by other sensors/measures including, temperature, color, EMG, etc. The adaptive circuit, disclosed above, can be used to adjust parameters based on the pH measurements.

Parameters of both the compounds and the skin can be measured/sensed, leading to subsequent electrical stimulation parameter adjustments. For example, for the skin, sensors can be used to gauge skin thickness, spO2 saturation (degree of vascularization), temperature (degree of blood flow), skin elasticity (ultrasound techniques), UVA/UVB, color, pH, skin biome and flora, where imbalances in one or more of these characteristics can lead to dysbiosis such as common disorders of acne and eczema (atopic dermatitis), depth of penetration of the electrical stimulation, etc. For the formulations, sensors are used to gauge the pH level, color, inflammation, etc.

Based on the sensed data of both the skin and the formulations, example inventions use electrical stimulation to revise the pH levels of the formulation/compound and the skin so that the pH levels of both approximately match (or come closer to matching than without the application of the electrical stimulation. For example, the formulation of Aloe Vera is a large complex molecule. Cosmetics generally count on it being broken down into smaller components but not to the micelle level for better absorption by the skin. Example inventions “reformulate” the formulations using electrical stimulation, and uses sensors to determine what chemical pathways/products are being formed.

Specifically, in example inventions, the pH level of a compound/formulation (e.g., Aloe Vera, Vitamin C, Vitamin A, etc.) that is either incorporated into patch 110 with the Hydrogel, or applied separately to the surface of the skin is measured. Further, the pH level of the skin is measured. The pH levels can be measured using sensors 115 using conductivity measurements and calculations. Further, in some examples, the pH levels can be measured using an ISFET that measures the pH of a solution (i.e., the formulation) and body fluids such as sweat. Electrical stimulation can then be used to change the pH level of the formulation and/or skin so that they approximately match, which enhances the ability of the formulation to enter the body through the skin, or the pH of the formulation is adjusted to some other target pH level.

In other examples, electrical stimulation can be used to alter the chemical composition of the formulation such as cosmetic lotion/serum. For example, the formulation may include a precursor to Vitamin A, and the electrical stimulation can change the chemical bonds of the precursor to convert the precursor into another form of Vitamin A. As disclosed above, the pH level of the formulation can also be altered. Sensors 115 can also be used to measure the chemical composition of a formulation, including spectroscopy or microfluidic chips that incorporate chemical assay techniques. In another example, minoxidil can be chemically altered by the electrical stimulation.

In examples, the Body Area Network disclosed above can be coupled to devices such as fitness devices and other wearables, cardio measurement devices, training devices such as a Peloton, etc. The data from these devices can be used to modify electrical stimulation parameters or formulation parameters.

As disclosed, system 102 uses electrical stimulation to promote alopecia repair. The alopecia repair includes, in response to the electrical stimulation, first order effects including increased blood flow in response to vasodilation. The increased blood flow is critical to all forms of healing and collagen production (e.g., wound healing, wrinkle repair, bone growth, etc.). The first order effects further include the upregulation of Vascular Endothelial Growth Factor (“VEGF”) which is a precursor to angiogenesis, and angiogenesis itself because the electrical stimulation increases vascularization by creating new blood vessels, which in turn increases the net flow of blood to hair follicles.

An additional first order effect is the electrical stimuli exercising the arrector pili muscle (“APM”) to maintain follicular stability and integrity, and help prevent loss of attachment to the primary follicles and other secondary follicles in the group of terminal hairs associated with each APM (generally 3-5 terminal hairs, there is an hierarchy of terminal hairs in a follicular unit, where the secondary hairs miniaturize first). There are other factors involved in the detachment of the APM from the hair follicle such as alterations in the hair cycle phases (ratio of anagen/telogen times), miniaturization, inflammation, hormone levels, and others including genetic factors. The electrical stimuli will counter or postpone many of these pro-alopecia factors. Additionally, by stimulating the APM during the treatment for alopecia, the electrical stimuli will go beyond minoxidil's capabilities and help to maintain the health of the hair follicle.

In addition, electrical stimuli keep the follicle unit functioning and prevent APM detachment and the creation of single-hair follicle. Selective miniaturization can lead to a reduction in the number of terminal hairs per follicular unit and the reduction in the number of hairs per follicular unit is associated with diffuse hair loss in women (i.e., areta alopecia).

The alopecia repair further includes second order effects such as iontophoresis (discussed above) and reinnervation that keeps the hair follicle healthy by maintaining the nerve plexus in the dermis and around the follicle bulb. Reinnevation helps to repair any damaged nerves surrounding follicles and help to maintain healthy ones.

The use of patch 110 enables these mechanisms by being adaptable in form factor to varying alopecia geometries. Given the form factor and the electrode arrangement, treatment regimens can be automatically adjusted for alopecia geometries upon initial application as well as downstream as the environment changes (e.g., changing impedances, degree of user comfort, etc.). Further, patch 110 can enable one or more electrodes in various configurations, such as a simple pair across the follicle repair area, an array surrounding the follicle repair area, or linear arrays at each side of the follicle repair area. Further, patch 110 can vary the treatment over time (e.g., pulsing vs DC, relative intensity over time, etc.). Further, patch 110 can vary treatment durations from minutes to hours to days, depending upon available power sources, which may vary from 100 mAH to thousands of mAH. Further, patch 110 can adjust treatment upon inputs from sensors. Patch 110 can take into account direct observations from a healthcare professional or the user, whose recommendations can be entered into the treatment regimen through a smart controller. Feedback can also be obtained directly from patch 110 and sent to the smart controller and in turn sent remotely so that a 3^{rd} party can make any necessary adjustments remotely.

Further, in addition to alopecia repair, example inventions can improve skin health because there are specific bacteria on the skin that are correlated with the occurrence of acne (i.e., Propionibacterium acnes) and eczema/atopic dermatitis (i.e., Staphylococcus aureus). Electrical stimulation applied by patch 110 can help to control or ameliorate the presence of these bacteria. Further, example inventions can lead to a better appearance due to other factors besides elimination of follicle loss. For example, electrical stimulation applied by patch 110 can assist in achieving a “glow” and “radiance” (i.e., actual measures used in the beauty industry that are measured by optical means such a reflected facial radiation at different wavelengths) by tightening the skin, smoothing it out, decreasing pore size, and other effects.

Several examples are specifically illustrated and/or described herein. However, it will be appreciated that modifications and variations of the disclosed examples are covered by the above teachings and within the purview of the appended claims without departing from the spirit and intended scope of the invention.

What is claimed is:

1. A method of reducing hair loss in a target follicle area where the hair loss has occurred, the method comprising:
affixing a patch externally on a dermis of a user so that the patch extends over the target follicle area of the user, the patch comprising a flexible substrate, a processor directly coupled to the substrate, and electrodes directly coupled to the substrate; and
activating the patch to initiate a treatment session, the activating comprising generating electrical stimuli via the electrodes that is directed at the target follicle area.

2. The method of claim 1, the electrical stimuli comprising a series of pulses comprising a pulse frequency between approximately 2 Hz and 150 Hz, and an applied current of up to approximately 100 milliamps.

3. The method of claim 1, the electrical stimuli comprising a series of pulses with a pattern comprising an intensity and a duration, the method comprising a plurality of the treatment session, further comprising monitoring a progress of a repairing of the hair loss and adjusting the intensity or the duration of the pattern during or between each treatment session based on the progress.

4. The method of claim 3, the monitoring comprising determining a state of at least one hair follicle and corresponding arrector pili muscle (APM) during or between treatment sessions.

5. The method of claim 3, the patch further comprising one or more sensors, the monitoring using the sensors.

6. The method of claim 5, the sensors comprising one or more of: a pH sensor, a photo sensor, an electrical sensor, an electromyography sensor (EMG) sensor, a gas detection sensor, an acoustic sensor, or a thermal sensor.

7. The method of claim 1, the patch comprising a hydrogel layer, the hydrogel layer including a compound for iontophoretic transfer.

8. The method of claim 7, the electrical stimulation altering a chemical composition of the compound.

9. The method of claim 8, the altering comprising molecular or structural changes to the compound that will modify its composition and/or function.

10. The method of claim 7, further comprising:
determining a first pH level of the dermis;
determining a second pH level of the compound; and
generating the electrical stimuli to modify the first pH level and/or the second pH level so that modified first pH level and the modified second pH level are approximately equal.

11. The method of claim 1, further comprising measuring a thickness of the dermis.

12. The method of claim 11, further comprising adjusting a charge profile based on the measured dermis thickness.

13. An alopecia repair system for reducing hair loss in a target follicle area where the hair loss has occurred, the system comprising:
a patch adapted to be externally coupled on a dermis of a user so that the patch extends over the target follicle area of the user, the patch comprising a flexible substrate, a processor directly coupled to the substrate, and electrodes directly coupled to the substrate; and
the processor adapted to activate the patch to initiate a treatment session, the activating comprising generating electrical stimuli via the electrodes that is directed at the target follicle area.

14. The system of claim 13, the electrical stimuli comprising a series of pulses comprising a pulse frequency between approximately 2 Hz and 150 Hz, and an applied current of up to approximately 100 milliamps.

15. The system of claim 13, the electrical stimuli comprising a series of pulses with a pattern comprising an intensity and a duration, the processor adapted to generate a plurality of the treatment sessions, further comprising monitoring a progress of a repairing of the hair loss and adjusting the intensity or the duration of the pattern during or between each treatment session based on the progress.

16. The system of claim 15, the monitoring comprising determining a state of at least one hair follicle and corresponding arrector pili muscle (APM) during or between treatment sessions.

17. The system of claim 15, the patch further comprising one or more sensors, the monitoring using the sensors.

18. The system of claim 17, the sensors comprising one or more of: a pH sensor, a photo sensor, an electrical sensor, an electromyography sensor (EMG) sensor, a gas detection sensor, an acoustic sensor, or a thermal sensor.

19. The system of claim 13, the patch comprising a hydrogel layer, the hydrogel layer including a compound for iontophoretic transfer.

20. The system of claim 19, the electrical stimulation altering a chemical composition of the compound.
